(19) United States
(12) Patent Application Publication
(10) Pub. No.: US 2026/0347450 A1
(43) Pub. Date: Nov. 5, 2026

(54) ENGINEERED CELLS TO DETECT AND RESPOND TO AMYLOID BETA PEPTIDES

(71) Applicant: Vanderbilt University, Nashville, TN (US)

(72) Inventors: Jonathan M. Brunger, Jr., Nashville, TN (US); Madeline R. Spetz, Nashville, TN (US); Ethan S. Lippmann, Nashville, TN (US)

(73) Assignee: Vanderbilt University, Nashville, TN (US)

(21) Appl. No.: 19/488,832

(22) PCT Filed: May 28, 2024
(86) PCT No.: PCT/US2024/031260

§ 371 Date: Nov. 30, 2025

Related U.S. Application Data
(60) Provisional application No. 63/505,606, filed on Jun. 1, 2023.

(51) Int. Cl. C07K 16/18; A61K 35/28; A61K 35/30; A61K 35/33; A61K 45/06; C07K 14/435; C07K 14/475; C07K 14/705; C07K 14/715; C12N 5/077; C12N 5/0775; C12N 5/079

(52) U.S. Cl. CPC C07K 16/18; A61K 35/28; A61K 35/30; A61K 35/33; A61K 45/06; C07K 14/43595; C07K 14/475; C07K 14/705; C07K 14/70503; C07K 14/70578; C07K 14/7155; C12N 5/0622; C12N 5/0656; C12N 5/0668; C07K 2317/21; C07K 2317/24; C07K 2317/622; C07K 2319/03; C12N 2510/00

(57) ABSTRACT

The present disclosure is directed to engineered cells designed to sense surface bound amyloid-P and to express one or more proteins in response to this binding event. In addition, the cells and associated methods of use can detect, treat, and recapitulate the symptoms of Alzheimer's disease. The engineered cells can regulate expression of neuronal growth factors and anti-inflammatory proteins to address neurodegeneration and neuroinflammation, respectively.

### PRIORITY CLAIM

This application claims benefit of priority to U.S. Provisional Application Ser. No. 63/505,606, filed Jun. 1, 2023, the entire contents of which are hereby incorporated by reference.

### BACKGROUND

### 1. Field of the Disclosure

The present disclosure relates generally to the fields of medicine, neurology, and molecular biology. More particularly, the disclosure relates to the development of engineered cells that are able to bind to amyloid-62 and, when bound, deliver diagnostic signals or therapeutic responses.

### 2. Background

Putative etiologies for AD include the interactive factors of amyloid deposition, tau propagation, metabolic dysfunction, vascular pathology, and neuroinflammation (Knopman et al., 2021; Butterfield & Halliwell, 2019; Baik et al., 2019; Tonnies & Trushina, 2017). To date, efforts to selectively target one of these leads to deleterious consequences pertaining to another. One striking example is the treatment of AD with amyloid-clearing monoclonal antibodies, which is associated with increased incidence of edema and microhemorrhage (Tolar et al., 2020; Sperling et al., 2012; Salloway et al., 2022), reflecting treatment-associated exacerbation of microvascular pathology (Xiong et al., 2021). Thus, a major question is how to combat AD without further disrupting brain homeostasis.

One approach is the develop of engineered cells that not only act as sophisticated drug delivery vehicles but do so by intelligently surveying their microenvironment and activating spatiotemporally restricted functionalities “on demand” to resolve selected aspects AD pathology. Over the past fifteen years, cell-based therapies, including induced pluripotent stem cell (iPSC)-derived neural stem cells and mesenchymal stem cells, have received increasing interest as multimodal engines to treat AD (Liu et al., 2020; Blurton-Jones et al., 2009; Chen & Blurton-Jones, 2012). While results continue to encourage activity in this domain, preclinical and clinical outcomes have demonstrated challenges associated with reliability of cell transplants for AD treatment (Blurton-Jones et al., 2009; Chen & Blurton-Jones, 2012). Unfortunately, trials evaluating transplanted cells' phenotypic fate, their ability to secrete neurotrophic factors, and their propensity to localize to effectively resolve AD pathology have met with disappointment (Marsh et al., 2017).

To improve reliability and reproducibility, the field has focused on illuminating the mechanisms of purported action of cells and designing methods to better coordinate the production of neurotrophic factors (including via genetic engineering of native cells) (Nagahara et al., 2018), improvement of neuronal synaptic connectivity, and specification of desired phenotypes of transplanted cells (Liu et al., 2020; Marsh et al., 2017; Shen et al., 2017; Tan et al., 2012; Bagheri-Mohammadi, 2021). By using synthetic biology to attack this problem, it may be possible to rationally assemble new biological systems that possess predictable and finely controlled properties. Indeed, cell therapies in other areas have resulted in remarkable outcomes in certain medical specialties. One noteworthy example is the generation of chimeric antigen receptors (CARs) that allow oncologists to re-target T cells to eradicate previously intractable tumors (Lim & June, 2017; June & Sadelain, 2018). A similar approach to treating AD with cells programmed to specifically attenuate pathological features of the neurodegenerative niche holds great, but as of yet unmet potential.

### SUMMARY

Thus, in accordance with the present disclosure, there is provided engineered cell expressing (a) a synthetic receptor expressed on the engineered cell's surface and comprising:
(1) an extracellular domain which serves as a recognition motif capable of engaging amyloid beta (A);
(2) a combination juxtamembrane/transmembrane domain which anchors the receptor in the cell membrane and contains protease cleavage sites that are exposed after receptor conformational changes take place in response to Aβ binding; and
(3) an intracellular domain that, upon Aβ binding and subsequent protease-based cleavage of the receptor, is untethered from the receptor, and
(b) a protein that is operable linked to a control sequence that is activated upon receptor binding to Aβ. The synthetic receptor may comprise transmembrane domain, such as one selected from Notch4, SORT1, Notch1, PCDHGC3, APP, CLSTN1, NOTCH2, NOTCH3, CLSTN2, NECTIN1, and EPCAM, and a juxtamembrane domain, such as one selected from NRG1, CSF1R, NOTCH3, NOTCH1, KCNE3, AGER, NOTCH4, PTPRF, PTPRM, NOTCH2, NRG2, LRP1B, PTPRF, JAG2, KL, EPHA4, PTPRK, CDH5, NOTCH1 and DAG1. The sense and response platform may be synNotch, such as where a Notch extracellular domain is replaced with a heterologous recognition motif and the Notch intracellular domain is replaced with a selected transcription factor, such as Ga14VP64, the tetracycline transactivator, or mammalian-derived transcriptional domains, such as zinc finger transcription factors. Alternatively, the sense and response platform is SyNthetic Intramembrane Proteolysis Receptor (SNIPR), where the extracellular heterologous recognition motif and the intracellular transcription factor are connected by truncated Notch transmembrane and juxtacrine domains. The engineered cell may be a fibroblast, a mesenchymal stem cell, an astrocyte, a microglial cell, a neuron, a neural stem cell, a macrophage, a T cell or a microvascular endothelial cell. The extacellular domain may be an antigen variable domain, such as may be located in a nanobody or single chain Fv (scFv). The scFv may be derived from the variable domain of an antibody selective for parenchymal versus vascular Aβ, an antibody selective for fibrillar Aβ or higher ordered aggregates, an antibody selective for Aβ plaques, or an antibody selective for fibrillar Aβ or higher ordered aggregates. The protein may be a detectable marker protein, such as Green Fluorescent Protein, mCherry, Blue fluorescent protein, infrared fluorescent protein, mCardinal, firefly luciferase, nanoluciferase, a BRET reporter (e.g., lumifluor), a FRET reporter, secreted alkaline phosphatase, gaussian luciferase, Renilla luciferase, or a MRI reporter (OATP1B3 transporter). The protein may be a therapeutic protein, such as brain-derived neurotrophic factor (BDNF), presenilin, anti-inflammatory proteins such as interleukin-10, transforming growth factor beta 1 (TGFβ1), fibroblast growth factor (FGF), native inhibitors of interleukin-1, (e.g., IL1Ra) interleukin-6, or tumor necrosis factor (e.g., sTNFR1) or biologic neutralizing factors (canakinumab, ziltivekimab, infliximab, adalimumab); inhibitors of complement or a cascade factor; an amyloid clearing factor (neprilysin, anti-Aβ antibody); or an anti-tau factor (e.g., bepranemab). The protein may be one that promotes an Alzheimer's Disease phenotype in said cell, such as TREM2, CD33, APOE, SERPINA3, CLU, BIN1, SORL1, ABCA7, ADAM10, APP, PSEN1, or IGHG3.

Also provided is a method of preventing the onset of, inhibiting the progression of or treating Alzheimer's Disease (AD) comprising administering to a subject in need thereof an engineered cell as defined herein. The subject may have been diagnosed with AD, such as by behavioral testing or by a brain scan, or have been determined to be at elevated risk of developing AD as compared to a populational average, such as by genetic testing or by familial history. The subject may be over 40 years of age, over 50 years of age, over 60 years of age, over 70 years of age, or over 80 years of age. The method may further comprise administering said engineered cell to said subject more than once. The subject may have been administered said engineered cell 2, 3, 4, 5, 6, 7, 8, 9, 10 15, or 20 times. The subject may be administered said engineered cell every week, every two weeks, every three weeks, month, every other month, every three months, every four months, every six months or every year. The subject may be treated with at least a second anti-AD therapy the reduces risk of developing AD and/or reduces symptoms of AD.

Yet further provided is a method of assessing or monitoring amyloid accumulation in subject comprising administering to said subject an engineered cell as defined herein. The subject may have been diagnosed with AD, such as by behavioral testing or by a brain scan, or have been determined to be at elevated risk of developing AD as compared to a populational average, such as by genetic testing or by familial history. The subject may be over 40 years of age, over 50 years of age, over 60 years of age, over 70 years of age, or over 80 years of age. The method may further comprise administering said engineered cell to said subject more than once. The subject may have been administered said engineered cell 2, 3, 4, 5, 6, 7, 8, 9, 10 15, or 20 times. The subject may be administered said engineered cell every week, every two weeks, every three weeks, month, every other month, every three months, every four months, every six months or every year. The subject may be treated with at least an anti-AD therapy the reduces risk of developing AD and/or reduces symptoms of AD.

In still a further embodiment, there is provided one or more expression vectors expressing (a) a synthetic receptor:
(1) an extracellular domain which serves as a recognition motif capable of engaging Aβ;
(2) a combination juxtamembrane/transmembrane domain which anchors the receptor in the cell membrane and contains protease cleavage sites that are exposed after receptor conformational changes take place in response to Aβ binding; and
(3) an intracellular domain that, upon Aβ binding and subsequent protease-based cleavage of the receptor, is untethered from the receptor, and
(b) a protein that is operably linked to a control sequence that is activated upon receptor binding to Aβ. The synthetic receptor may comprise a transmembrane domain, such as one selected from Notch4, SORT1, Notch1, PCDHGC3, APP, CLSTN1, NOTCH2, NOTCH3, CLSTN2, NECTIN1, and EPCAM, and a juxtamembrane domain, such as one selected from NRG1, CSFIR, NOTCH3, NOTCH1, KCNE3, AGER, NOTCH4, PTPRF, PTPRM, NOTCH2, NRG2, LRP1B, PTPRF, JAG2, KL, EPHA4, PTPRK, CDH5, NOTCH1 and DAG1. The sense and response platform may be synNotch, such as where a Notch extracellular domain is replaced with a heterologous recognition motif and the Notch intracellular domain is replaced with a selected transcription factor. The selected transcription factor may be Ga14VP64, the tetracycline transactivator, or a mammalian-derived transcriptional domain, such as a zinc finger transcription factor. The sense and response platform may be Synthetic Intramembrane Proteolysis Receptor (SNIPR).

The expression vector(s) of claim 31, wherein the extracullular domain is:
(i) an antigen variable domain, such as is located in a nanobody or single chain Fv (scFv); and/or
(ii) the extracellular domcina is an scFv derived from the variable domain of an antibody selective for parenchymal versus vascular Aβ, an antibody selective for fibrillar Aβ or higher ordered aggregates, an antibody selective for Aβ plaques, or an antibody selective for fibrillar Aβ or higher ordered aggregates.

The protein may be a detectable marker protein, such as wherein the detectable marker protein is Green Fluorescent Protein, mCherry, Blue fluorescent protein, infrared fluorescent protein, mCardinal, firefly luciferase, nanoluciferase, a BRET reporter (e.g., lumifluor), a FRET reporter, secreted alkaline phosphatase, gaussian luciferase, or a MRI reporter (OATP1B3 transporter). The protein may be a therapeutic protein, such as brain-derived neurotrophic factor (BDNF), presenilin, anti-inflammatory proteins such as interleukin-10, transforming growth factor β1 (TGFβ1), fibroblast growth factor, native inhibitors of interleukin-1, (e.g., IL1Ra) interleukin-6, or tumor necrosis factor (e.g., sTNFR1) or biologic neutralizing factors (canakinumab, ziltivekimab, infliximab, adalimumab); inhibitors of complement or a cascade factor; an amyloid clearing factor (neprilysin, anti-Aβ antibody); or an anti-tau factor (e.g., bepranemab). The protein may be a protein that can promote an Alzheimer's Disease phenotype in said cell, such as TREM2, CD33, APOE, SERPINA3, CLU, BIN1, SORL1, ABCA7, ADAM10, APP, PSEN1, or IGHG3.

Also provided is a method of preventing the onset of, inhibiting the progression of or treating Alzheimer's Disease (AD) comprising administering to a subject in need thereof a vector or vectors as described herein. The subject may have been diagnosed with AD, such as by behavioral testing or by a brain scan. The subject may have been determined to be at elevated risk of developing AD as compared to a populational average. The subject may have been determined to be at elevated risk of developing AD as compared to a populational average by genetic testing or by familial history. The subject may be over 40 years of age, over 50 years of age, over 60 years of age, over 70 years of age, or over 80 years of age. The method may further comprise administering said expression vector(s) to said subject more than once, such as 2, 3, 4, 5, 6, 7, 8, 9, 10 15, or 20 times, and/or wherein the subject is administered said expression vector(s) every week, every two weeks, every three weeks, month, every other month, every three months, every four months, every six months or every year. The subject may be treated with at least a second anti-AD therapy that reduces risk of developing AD and/or reduces symptoms of AD. The expression vector(s) may be viral vectors, such as an AAV vector, a lentiviral vector and an HSV vector. The expression vector(s) may be non-viral vectors, such a non-viral vector delivered in or with a lipid nanoparticle, porous silicon nanoparticle, polymer nanoparticles, or gold nanoparticles, dendrimers, carbon nanotubes.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1: Schematic of synthetic receptors that enable detection of amyloid-p. Amyloid recognition via the artificial signaling channel enables regulated expression of selected genes. Example recognition domains and regulated transgenes are illustrated.

FIG. 2: Responsiveness of engineered L929 fibroblasts expressing one of the artificial amyloid receptors to human brain-derived amyloid-β. Left: phase contrast; Center: blue fluorescence indicates cells expressing receptor platform elements; Right: fluorescence indicates cells expressing mCherry reporter induced by artificial receptor activation. Scale bar=200 microns.

FIG. 3: Fold luminescence change as a measure of receptor activation levels induced by immobilized, biotinylated Aβ42 (Anaspec) peptide by the three Aβ-sensitive synNotch receptors (Gant-, Don-, and Bap-Notch) and as compared to a GFP-sensitive synNotch receptor (LaG16). * p<0.05; ** p<0.01; *** p<0.001; **** p<0.0001.

FIG. 4: Fold luminescence change as a measure of receptor activation levels induced by Aβ42 peptide (Anaspec) either passively adsorbed (left) to a culture dish or added to culture medium (right). Results compare activation levels of the three Ab-sensitive synNotch receptors (Gant-, Don-, and Bap-Notch) to a GFP-sensitive synNotch receptor (LaG16). * p<0.05; ** p<0.01; **** p<0.0001.

FIG. 5: Fold luminescence change as a measure of receptor activation levels induced by immobilized, biotinylated Aβ₄₀ by the three Aβ-sensitive synNotch receptors (Gant-, Don-, and Bap-Notch) and as compared to a GFP-sensitive synNotch receptor (LaG16). * p<0.05; ** p<0.01; **** p<0.0001.

FIGS. 6A-C: Characterization of an Ad-Notch receptor. (FIG. 6A) Expression of Ad-Notch in L929 mouse fibroblasts, indicated by BFP and c-myc epitope expression (probed for by flow cytometry with a fluorescent anti-c-myc antibody). (FIG. 6B) mCherry fluorescence indicates activation of the Ad-Notch receptor. Activation here is generated by anti-myc antibody coated beads that interface with a c-myc epitope tag on the N-terminus of each synNotch receptor. Bottom: blue fluorescence indicates cells that express artificial receptor platform elements in the same field of view as Top. Scale bar=200 microns. (FIG. 6C) Ad-Notch does not recognize biotinylated synthetic Aβ₄₂. There is no fold induction in SynNotch-driven luciferase expression in Ad-Notch cells (compare to ~30-fold induction in Gant-Notch in the same experiment). **** p<0.0001.

FIG. 7: Responsiveness of engineered mesenchymal stem cells expressing the Gant-Notch artificial amyloid receptor to synthetic Ab42 (top) or control culture conditions (bottom). Microscopy-Left: blue fluorescence indicates cells expressing receptor platform elements; Center: fluorescence indicates cells expressing mCherry reporter induced by artificial receptor activation. Merge: indicates fluorescence in both blue and red channels. Scale bar=200 microns. Plot-raw luminescence values demonstrating Ab-dependent receptor activation of mesenchymal stem cells. * p<0.05 Student's t-test.

FIG. 8: Phase contrast and fluorescence microscopy indicating activation of Gant-Notch and Bap-Notch in H9 human embryonic stem cells by immobilization of biotinylated Aβ₄₂ peptide. Microscopy compares control (bottom) vs. Aβ42 treated culture surfaces.

FIG. 9: A murine L929 fibroblast cell line was engineered to express the Bap-Notch receptor, which was designed to regulate expression of a TREM2 transgene. Fluorescence microscopy indicates that engineered fibroblasts inducibly express the myeloid marker of TREM2 in response to Aβ treatment. Left: Draq5 nuclear dye shown in magenta; Center: TREM2 immunofluorescence shown in green; Merge: merging of the nuclear (magenta) and TREM2 (green) fluorescence signals. Scale bar=200 microns.

FIG. 10: Demonstration of the ability of an artificial Ab-synNotch receptor to regulate astrocyte transgene expression. Phase contrast and mCherry fluorescence microscopy indicates the Aβ-dependent regulation of mCherry transgene expression in Gant-Notch and Bap-Notch engineered human iPS-derived astrocytes. Inset shows GFAP staining of human iPS-derived astrocytes, confirming cellular phenotype. Scale bar=200 microns.

FIG. 11: Demonstration of the ability of Aβ-synNotch receptor to regulate BDNF transgene expression. Human iPSC-derived astrocytes were engineered to express either brain derived neurotrophic factor (BDNF) or mCherry upon synNotch activation. Bap-Notch cells engineered with a synNotch-regulated BDNF transgene exhibited amyloid-dependent upregulation of BDNF as measured by ELISA, whereas cells engineered to regulate mCherry via synNotch did not. **** p<0.0001.

FIG. 12: Attenuation of A1 reactive astrocyte phenotype by SynNotch-driven anti-inflammatory transgenes. Human iPSC-derived astrocytes were engineered to express soluble TNF receptor (sTNFr) and IL-1Ra upon SynNotch activation. Bap-Notch astrocytes were induced to take on a reactive phenotype through addition of TNF-α and IL-1α, as indicated by expression of representative reactive genes Il6, CSF, SERPINA3, and C3. When the astrocytes are also plated on synthetic Aβ₄₂, the expression of sTNFr and IL-1Ra antagonizes the inflammatory cytokines and results in a significant decrease in the expression of Il6, CSF2, and SERPINA3. No significant difference in the expression of C3. Bap-Notch astrocytes engineered to express a control SEAP transgene show no difference in expression levels of reactive genes. *p<0.05; ****p<0.0001.

FIG. 13: Validation of a Bap-SNIPR receptor. Fold luminescence change as a measure of receptor activation levels induced by immobilized, biotinylated Aβ42 for Bap-SNIPR compared to Bap-Notch in mMSCs.

### DESCRIPTION OF ILLUSTRATIVE EMBODIMENTS

As discussed above, accumulating evidence from transgenic animal models and post-mortem human studies impute aberrant microglial and astrocytic states as causal drivers of neurodegeneration in Alzheimer's disease (AD). For example, murine models have demonstrated that dramatic microglial and astrocytic phenotypic transitions occur in association with Aβ plaque deposition, while studies on human brain tissue suggest a link between plaque accumulation, neurite deterioration, and gene expression patterns. However, the causal relationships between microglial/astrocyte activity, Aβ accumulation and disease progression are not well characterized in human studies. Due to differences in gene expression modules between rodents and humans, and the inaccessibility of the human brain to prospective in vivo investigations, the field lacks a thorough understanding of how human phenotypic changes propagate causative elements of the neurodegenerative niche.

Here, the inventors employed a synthetic biology tool known as synNotch, which is described in numerous scientific publications (e.g., Morsut et al., Cell 2016, Roybal et al., Cell 2016a; Roybal et al., Cell 2016b). By exchanging Notch's (1) extracellular domain with alternative recognition motifs (e.g., single chain variable fragments (scFvs)) and (2) intracellular domain with a synthetic transcription factor, the inventors created synNotch receptors that produce user-specified sense/response behaviors. The inventors also employ a synthetic biology tool that is a further innovation of SynNotch-like intramembrane proteolysis receptors known as SNIPR (Synthetic Intramembrane Proteolysis Receptor) (Zhu et al., Cell 2022). Using these platforms, or comparable ones, the inventors can produce customized responses to Aβ inputs, both for better understanding of the disease state itself and driving cells to implement putative neurodegenerative or protective gene expression programs.

Uniquely, the methods described herein enable customization of cell behaviors in response to amyloid-beta inputs. While β-amyloidosis is a biochemical hallmark of Alzheimer's disease (AD), many distinct forms of Aβ, including soluble monomers, soluble oligomers, insoluble fibrils, and plaques, are thought to be causative agents in AD onset and progression. And it is thought that small Aβ oligomers may be more neurotoxic than amyloid-fibrils by templating the formation of pathologic tau neurofibrillary tangles, while higher ordered assemblies such as plaques induce the emergence of various disease-associated phenotypes. The ability to engineer cells to selectively detect various species of amyloid-β opens the possibility to customize defined cellular responses to abrogate context-specific aspects of AD pathology. Unlike biologic drugs, cells can integrate multiple inputs to perform an array of outputs as diverse as the assembly of neotissue, production of multiple anti-inflammatory agents or engulfment of foreign bodies. As such, cells can serve as exceptional agents to coordinate regeneration and combat disease.

Thus, taking advantage of the linkage between a cell's microenvironmental niche and customized signaling responses can be used to study the causal relationship between amyloid inputs and putatively pathologic cellular behaviors, greatly advancing understanding of the disease state. Moreover, anti-amyloid-β and anti-tau immunotherapies have been pursued as targets for treating AD, such biologic drugs do not permit customizable responses that can work to simultaneously antagonize multiple aspects of the disease in a self-regulatable manner. As such, cell-based therapeutics like those described here are more likely to successfully modulate amyloid-β pathology, potentially offering a tremendous breakthrough in AD therapy.

These and other aspects of the disclosure are described in detail below.

### I. Alzheimer's Disease and the Role of Amyloid Beta

### A. Alzheimer's Disease

Alzheimer's disease (AD) is a neurodegenerative disease that usually starts slowly and progressively worsens. It is the cause of 60-70% of cases of dementia. The most common early symptom is difficulty in remembering recent events. As the disease advances, symptoms can include problems with language, disorientation (including easily getting lost), mood swings, loss of motivation, self-neglect, and behavioral issues. As a person's condition declines, they often withdraw from family and society. Gradually, bodily functions are lost, ultimately leading to death. Although the speed of progression can vary, the typical life expectancy following diagnosis is three to nine years.

The cause of Alzheimer's disease is poorly understood. There are many environmental and genetic risk factors associated with its development. The strongest genetic risk factor is from an allele of APOE. Other risk factors include a history of head injury, clinical depression, and high blood pressure. The disease process is largely associated with amyloid plaques, neurofibrillary tangles, and loss of neuronal connections in the brain. A probable diagnosis is based on the history of the illness and cognitive testing with medical imaging and blood tests to rule out other possible causes. Initial symptoms are often mistaken for normal brain aging. Examination of brain tissue is needed for a definite diagnosis, but this can only take place after death. Good nutrition, physical activity, and engaging socially are known to be of benefit generally in aging, and these may help in reducing the risk of cognitive decline and Alzheimer's; in 2019 clinical trials were underway to look at these possibilities. There are no medications or supplements that have been shown to decrease risk. No treatments stop or reverse its progression, though some may temporarily improve symptoms. Affected people increasingly rely on others for assistance, often placing a burden on the caregiver. The pressures can include social, psychological, physical, and economic elements. Exercise programs may be beneficial with respect to activities of daily living and can potentially improve outcomes. Behavioral problems or psychosis due to dementia are often treated with antipsychotics, but this is not usually recommended, as there is little benefit and an increased risk of early death.

As of 2020, there were approximately 50 million people worldwide with Alzheimer's disease. It most often begins in people over 65 years of age, although up to 10% of cases are early-onset affecting those in their 30s to mid-60s. It affects about 6% of people 65 years and older, and women more often than men. The disease is named after German psychiatrist and pathologist Alois Alzheimer, who first described it in 1906. Alzheimer's financial burden on society is large, with an estimated global annual cost of US$1 trillion. Alzheimer's disease is currently ranked as the seventh leading cause of death in the United States.

The course of Alzheimer's is generally described in three stages, with a progressive pattern of cognitive and functional impairment. The three stages are described as early or mild, middle or moderate, and late or severe. The disease is known to target the hippocampus which is associated with memory, and this is responsible for the first symptoms of memory impairment. As the disease progresses so does the degree of memory impairment. Proteins fail to function normally. This disrupts the work of the brain cells affected and triggers a toxic cascade, ultimately leading to cell death and later brain shrinkage.

Alzheimer's disease is believed to occur when abnormal amounts of amyloid beta (AB), accumulating extracellularly as amyloid plaques and tau proteins, or intracellularly as neurofibrillary tangles, form in the brain, affecting neuronal functioning and connectivity, resulting in a progressive loss of brain function. This altered protein clearance ability is age-related, regulated by brain cholesterol, and associated with other neurodegenerative diseases.

Advances in brain imaging techniques allow researchers to see the development and spread of abnormal amyloid and tau proteins in the living brain, as well as changes in brain structure and function. Beta-amyloid is a fragment of a larger protein. When these fragments cluster together, a toxic effect appears on neurons and disrupts cell-to-cell communication. Larger deposits called amyloid plaques are thus further formed.

Tau proteins are responsible in neuron's internal support and transport system to carry nutrients and other essential materials. In Alzheimer's disease, the shape of tau proteins is altered and thus organize themselves into structures called neurofibrillary tangles. The tangles disrupt the transport system and are toxic to cells.

The cause for most Alzheimer's cases is still mostly unknown, except for 1-2% of cases where deterministic genetic differences have been identified. Several competing hypotheses attempt to explain the underlying cause; the two predominant hypotheses are the amyloid beta (Aβ) hypothesis and the cholinergic hypothesis.

The oldest hypothesis, on which most drug therapies are based, is the cholinergic hypothesis, which proposes that Alzheimer's disease is caused by reduced synthesis of the neurotransmitter acetylcholine. The loss of cholinergic neurons noted in the limbic system and cerebral cortex, is a key feature in the progression of Alzheimer's. The 1991 amyloid hypothesis postulated that extracellular amyloid beta (Aβ) deposits are the fundamental cause of the disease. Support for this postulate comes from the location of the gene for the amyloid precursor protein (APP) on chromosome 21, together with the fact that people with trisomy 21 (Down syndrome) who have an extra gene copy almost universally exhibit at least the earliest symptoms of Alzheimer's disease by 40 years of age. A specific isoform of apolipoprotein, APOE4, is a major genetic risk factor for Alzheimer's disease. While apolipoproteins enhance the breakdown of beta amyloid, some isoforms are not very effective at this task (such as APOE4), leading to excess amyloid buildup in the brain.

Alzheimer's disease is characterized by loss of neurons and synapses in the cerebral cortex and certain subcortical regions. This loss results in gross atrophy of the affected regions, including degeneration in the temporal lobe and parietal lobe, and parts of the frontal cortex and cingulate gyrus. Degeneration is also present in brainstem nuclei particularly the locus coeruleus in the pons. Studies using MRI and PET have documented reductions in the size of specific brain regions in people with Alzheimer's disease as they progressed from mild cognitive impairment to Alzheimer's disease and in comparison with similar images from healthy older adults.

Both Aβ plaques and neurofibrillary tangles are clearly visible by microscopy in brains of those with Alzheimer's disease, especially in the hippocampus. However, Alzheimer's disease may occur without neurofibrillary tangles in the neocortex. Plaques are dense, mostly insoluble deposits of beta-amyloid peptide and cellular material outside and around neurons. Tangles (neurofibrillary tangles) are aggregates of the microtubule-associated protein tau which has become hyperphosphorylated and accumulates inside the cells themselves. Although many older individuals develop some plaques and tangles as a consequence of aging, the brains of people with Alzheimer's disease have a greater number of them in specific brain regions such as the temporal lobe. Lewy bodies are not rare in the brains of people with Alzheimer's disease.

Alzheimer's disease has been identified as a protein misfolding disease (proteopathy) caused by the accumulation of abnormally folded amyloid beta protein into amyloid plaques, and tau protein into neurofibrillary tangles in the brain. Plaques are made up of small peptides, 39-43 amino acids in length, called amyloid beta (AB). Amyloid beta is a fragment from the larger amyloid-beta precursor protein (APP) a transmembrane protein that penetrates the neuron's membrane. APP is critical to neuron growth, survival, and post-injury repair. In Alzheimer's disease, gamma secretase and beta secretase act together in a proteolytic process which causes APP to be divided into smaller fragments. One of these fragments gives rise to fibrils of amyloid beta, which then form clumps that deposit outside neurons in dense formations known as amyloid plaques.

Alzheimer's disease is also considered a tauopathy due to abnormal aggregation of the tau protein. Every neuron has a cytoskeleton, an internal support structure partly made up of structures called microtubules. These microtubules act like tracks, guiding nutrients and molecules from the body of the cell to the ends of the axon and back. A protein called tau stabilizes the microtubules when phosphorylated and is therefore called a microtubule-associated protein. In Alzheimer's disease, tau undergoes chemical changes, becoming hyperphosphorylated; it then begins to pair with other threads, creating neurofibrillary tangles and disintegrating the neuron's transport system. Pathogenic tau can also cause neuronal death through transposable element dysregulation.

Exactly how disturbances of production and aggregation of the beta-amyloid peptide give rise to the pathology of Alzheimer's disease is not known. The amyloid hypothesis traditionally points to the accumulation of beta-amyloid peptides as the central event triggering neuron degeneration. Accumulation of aggregated amyloid fibrils, which are believed to be the toxic form of the protein responsible for disrupting the cell's calcium ion homeostasis, induces programmed cell death (apoptosis). It is also known that A_{β} selectively builds up in the mitochondria in the cells of Alzheimer's-affected brains, and it also inhibits certain enzyme functions and the utilization of glucose by neurons.

Various inflammatory processes and cytokines may also have a role in the pathology of Alzheimer's disease. Inflammation is a general marker of tissue damage in any disease and may be either secondary to tissue damage in Alzheimer's disease or a marker of an immunological response. There is increasing evidence of a strong interaction between the neurons and the immunological mechanisms in the brain. Obesity and systemic inflammation may interfere with immunological processes which promote disease progression.

Alterations in the distribution of different neurotrophic factors and in the expression of their receptors such as the brain-derived neurotrophic factor (BDNF) have been described in Alzheimer's disease.

### B. Aβ

Amyloid beta (Aβ or Abeta) denotes peptides of 36-43 amino acids that are the main component of the amyloid plaques found in the brains of people with Alzheimer's disease. The peptides derive from the amyloid precursor protein (APP), which is cleaved by beta secretase and gamma secretase to yield Aβ. Aβ molecules can aggregate to form flexible soluble oligomers which may exist in several forms. It is now believed that certain misfolded oligomers (known as “seeds”) can induce other Aβ molecules to also take the misfolded oligomeric form, leading to a chain reaction akin to a prion infection. These oligomers are toxic to nerve cells. The other protein implicated in Alzheimer's disease, Tau protein, also forms such prion-like misfolded oligomers, and there is some evidence that misfolded Aβ can induce Tau to misfold. A study has suggested that APP and its amyloid potential is of ancient origins, dating as far back as early deuterostomes.

The normal function of Aβ is not well understood. Though some animal studies have shown that the absence of Aβ does not lead to any obvious loss of physiological function, several potential activities have been discovered for Aβ, including activation of kinase enzymes, protection against oxidative stress, regulation of cholesterol transport, functioning as a transcription factor, and anti-microbial activity (potentially associated with Aβ's pro-inflammatory activity).

The glymphatic system clears metabolic waste from the mammalian brain, and in particular beta amyloids. Indeed, a number of proteases have been implicated by both genetic and biochemical studies as being responsible for the recognition and degradation of beta amyloids; these include insulin degrading enzyme and presequence protease The rate of removal is significantly increased during sleep. However, the significance of the lymphatic system in Aβ clearance in Alzheimer's disease is unknown.

Aβ is the main component of amyloid plaques, extracellular deposits found in the brains of people with Alzheimer's disease). Similar plaques appear in some variants of Lewy body dementia and in inclusion body myositis (a muscle disease), while Aβ can also form the aggregates that coat cerebral blood vessels in cerebral amyloid angiopathy. The plaques are composed of a tangle of regularly ordered fibrillar aggregates called amyloid fibers, a protein fold shared by other peptides such as the prions associated with protein misfolding diseases. As mentioned above, the toxic effect of Aβ-oligomers in the heart was reported in 2010 and the presence of Aβ in the heart of patients with Alzheimer's disease was reported in 2016.

Research suggests that soluble oligomeric forms of the peptide may be causative agents in the development of Alzheimer's disease. It is generally believed that Aβ oligomers are the most toxic. The ion channel hypothesis postulates that oligomers of soluble, non-fibrillar Aβ form membrane ion channels allowing the unregulated calcium influx into neurons that underlies disrupted calcium ion homeostasis and apoptosis seen in Alzheimer's disease. Computational studies have demonstrated that also Aβ peptides embedded into the membrane as monomers with predominant helical configuration, can oligomerize and eventually form channels whose stability and conformation are sensitively correlated to the concomitant presence and arrangement of cholesterol. A number of genetic, cell biology, biochemical and animal studies support the concept that Aβ plays a central role in the development of Alzheimer's disease pathology.

Brain Aβ is elevated in people with sporadic Alzheimer's disease. Aβ is the main constituent of brain parenchymal and vascular amyloid; it contributes to cerebrovascular lesions and is neurotoxic. It is unresolved how Aβ accumulates in the central nervous system and subsequently initiates the disease of cells. Some researchers have found that the AB oligomers induce some of the symptoms of Alzheimer's disease by competing with insulin for binding sites on the insulin receptor, thus impairing glucose metabolism in the brain. Significant efforts have been focused on the mechanisms responsible for Aβ production, including the proteolytic enzymes γ- and β-secretases which generate Aβ from its precursor protein, APP (amyloid precursor protein). Aβ circulates in plasma, cerebrospinal fluid (CSF) and brain interstitial fluid (ISF) mainly as soluble Aβ40 Senile plaques contain both Aβ40 and Aβ42, while vascular amyloid is predominantly the shorter Aβ40. Several sequences of Aβ were found in both lesions. Generation of Aβ in the central nervous system may take place in the neuronal axonal membranes after APP-mediated axonal transport of β-secretase and presenilin-1.

Increases in either total Aβ levels or the relative concentration of both Aβ40 and Aβ42 (where the former is more concentrated in cerebrovascular plaques and the latter in neuritic plaques) have been implicated in the pathogenesis of both familial and sporadic Alzheimer's disease. Due to its more hydrophobic nature, the Aβ42 variant is the most amyloidogenic form of the peptide. However, the central sequence KLVFFAE is known to form amyloid on its own, and probably forms the core of the fibril. One study further correlated Aβ42 levels in the brain not only with onset of Alzheimer's disease, but also reduced cerebrospinal fluid pressure, suggesting that a build-up or inability to clear Aβ42 fragments may play a role in the pathology.

The “amyloid hypothesis,” that plaques are responsible for the pathology of Alzheimer's disease, is accepted by the majority of researchers but is not conclusively established. An alternative hypothesis is that amyloid oligomers rather than plaques are responsible for the disease. Mice that are genetically engineered to express oligomers but not plaques (APP^{E693Q}) develop the disease. Furthermore, mice that are in addition engineered to convert oligomers into plaques (APP^{E693Q} X PS1ΔE9), are no more impaired than the oligomer only mice. Intra-cellular deposits of Tau protein are also seen in the disease, and may also be implicated, as has aggregation of alpha synuclein.

While Aβ has been implicated in cancer development, prompting studies on a variety of cancers to elucidate the nature of its possible effects, results are largely inconclusive. Aβ levels have been assessed in relation to a number of cancers, including esophageal, colorectal, lung, and hepatic, in response to observed reductions in risk for developing Alzheimer's disease in survivors of these cancers. All cancers were shown to be associated positively with increased Aβ levels, particularly hepatic cancers. This direction of association however has not yet been established. Studies focusing on human breast cancer cell lines have further demonstrated that these cancerous cells display an increased level of expression of amyloid precursor protein.

Adults with Down syndrome had accumulation of amyloid in association with evidence of Alzheimer's disease, including declines in cognitive functioning, memory, fine motor movements, executive functioning, and visuospatial skills.

Aβ is formed after sequential cleavage of the amyloid precursor protein (APP), a transmembrane glycoprotein of undetermined function. APP can be cleaved by the proteolytic enzymes α-, β- and γ-secretase; Aβ protein is generated by successive action of the β and γ secretases. The γ secretase, which produces the C-terminal end of the Aβ peptide, cleaves within the transmembrane region of APP and can generate a number of isoforms of 30-51 amino acid residues in length. The most common isoforms are Aβ₄₀ and Aβ₄₂; the longer form is typically produced by cleavage that occurs in the endoplasmic reticulum, while the shorter form is produced by cleavage in the trans-Golgi network.

Autosomal-dominant mutations in APP cause hereditary early-onset Alzheimer's disease (familial AD). This form of AD accounts for no more than 10% of all cases, and the vast majority of AD is not accompanied by such mutations. However, familial Alzheimer's disease is likely to result from altered proteolytic processing. The gene for the amyloid precursor protein is located on chromosome 21, and accordingly people with Down syndrome have a very high incidence of Alzheimer's disease.

Amyloid beta is commonly thought to be intrinsically unstructured, meaning that in solution it does not acquire a unique tertiary fold but rather populates a set of structures. As such, it cannot be crystallized and most structural knowledge on amyloid beta comes from NMR and molecular dynamics. Early NMR-derived models of a 26-aminoacid polypeptide from amyloid beta (Aβ 10-35) show a collapsed coil structure devoid of significant secondary structure content. However, the most recent (2012) NMR structure of (Aβ 1-40) has significant secondary and tertiary structure. Replica exchange molecular dynamics studies suggested that amyloid beta can indeed populate multiple discrete structural states; more recent studies identified a multiplicity of discrete conformational clusters by statistical analysis. By NMR-guided simulations, amyloid beta 1-40 and amyloid beta 1-42 also seem to feature highly different conformational states, with the C-terminus of amyloid beta 1-42 being more structured than that of the 1-40 fragment.

Low-temperature and low-salt conditions allowed to isolate pentameric disc-shaped oligomers devoid of beta structure. In contrast, soluble oligomers prepared in the presence of detergents seem to feature substantial beta sheet content with mixed parallel and antiparallel character, different from fibrils; computational studies suggest an antiparallel beta-turn-beta motif instead for membrane-embedded oligomers.

The suggested mechanisms by which amyloid beta may damage and cause neuronal death include the generation of reactive oxygen species during the process of its self-aggregation. When this occurs on the membrane of neurons in vitro, it causes lipid peroxidation and the generation of a toxic aldehyde called 4-hydroxynonenal which, in turn, impairs the function of ion-motive ATPases, glucose transporters and glutamate transporters. As a result, amyloid beta promotes depolarization of the synaptic membrane, excessive calcium influx and mitochondrial impairment. Aggregations of the amyloid-beta peptide disrupt membranes in vitro.

### II. Monoclonal Antibodies and Production Thereof

Antibodies to different forms of amyloid-β are one component of the present disclosure. Antibody production is an extremely well-developed technology and new antibodies are constantly being produced, including those that bind to Aβ. The following is a general discussion of antibody technology.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{II}) for each of the alpha and gamma chains and four CH domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in CH sequence and function, humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

### A. General Methods

Methods for preparing and characterizing antibodies are well known in the art (see, e.g., Antibodies: A Laboratory Manual, Cold Spring Harbor Laboratory, 1988; U.S. Pat. No. 4,196,265). The methods for generating monoclonal antibodies (MAbs) generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce Aβ-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

In the case of human antibodies against natural pathogens, a suitable approach is to identify subjects that have been exposed to the pathogens, such as those who have been diagnosed as having contracted the disease, or those who have been vaccinated to generate protective immunity against the pathogen or to test the safety or efficacy of an experimental vaccine. Circulating anti-pathogen antibodies can be detected, and antibody encoding or producing B cells from the antibody-positive subject may then be obtained.

The amount of immunogen composition used in the production of polyclonal antibodies varies with the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens, lymph nodes, tonsils or adenoids, bone marrow aspirates or biopsies, tissue biopsies from mucosal organs like lung or GI tract, or from circulating blood. The antibody-producing B lymphocytes from the immunized animal or immune human are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984). HMMA2.5 cells or MFP-2 cells are particularly useful examples of such cells.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986) and there are processes for better efficiency (Yu et al., 2008). Fusion procedures usually produce viable hybrids at low frequencies, about 1×10⁻⁶ to 1×10⁻⁸, but with optimized procedures one can achieve fusion efficiencies close to 1 in 200 (Yu et al., 2008). However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EB V-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 104 times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

### B. Antibodies of the Present Disclosure

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248:443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other band, if the test antibody is not able to bind to the target molecule following saturation binding with the reference antibody, then the test antibody may bind to the same epitope as the epitope bound by the reference antibody.

To determine if an antibody competes for binding with a reference anti-Aβ antibody, the above-described binding methodology is performed in two orientations: In a first orientation, the reference antibody is allowed to bind to Aβ under saturating conditions followed by assessment of binding of the test antibody to Aβ. In a second orientation, the test antibody is allowed to bind to Aβ molecule under saturating conditions followed by assessment of binding of the reference antibody to Aβ. If, in both orientations, only the first (saturating) antibody is capable of binding to Aβ, then it is concluded that the test antibody and the reference antibody compete for binding to Aβ. As will be appreciated by a person of ordinary skill in the art, an antibody that competes for binding with a reference antibody may not necessarily bind to the identical epitope as the reference antibody but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding, Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

### C. Engineering of Antibody Sequences

In various embodiments, one may choose to engineer sequences of the identified antibodies for a variety of reasons, such as improved expression, improved cross-reactivity or diminished off-target binding. Modified antibodies may be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. A PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. F(ab′) antibody derivatives are monovalent, while F(ab′)₂ antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e. g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23p19 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FeγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

FcRn binding. Fc mutations can also be introduced and engineered to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FeγRII, FcγRIII, and FcRn and design of IgG1 variants with improved binding to the FcγR, (J. Biol. Chem. 276:6591-6604). A number of methods are known that can result in increased half-life (Kuo and Aveson, (2011)), including amino acid modifications may be generated through techniques including alanine scanning mutagenesis, random mutagenesis and screening to assess the binding to the neonatal Fc receptor (FcRn) and/or the in vivo behavior. Computational strategies followed by mutagenesis may also be used to select one of amino acid mutations to mutate.

The present disclosure therefore provides a variant of an antigen binding protein with optimized binding to FcRn. In a particular embodiment, the said variant of an antigen binding protein comprises at least one amino acid modification in the Fc region of said antigen binding protein, wherein said modification is selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401 403, 404, 408, 411, 412, 414, 415, 416, 418, 419, 420, 421, 422, 424, 426, 428, 433, 434, 438, 439, 440, 443, 444, 445, 446 and 447 of the Fc region as compared to said parent polypeptide, wherein the numbering of the amino acids in the Fc region is that of the EU index in Kabat. In a further aspect of the disclosure the modifications are M252Y/S254T/T256E.

Additionally, various publications describe methods for obtaining physiologically active molecules whose half-lives are modified, see for example Kontermann (2009) either by introducing an FcRn-binding polypeptide into the molecules or by fusing the molecules with antibodies whose FcRn-binding affinities are preserved but affinities for other Fc receptors have been greatly reduced or fusing with FcRn binding domains of antibodies.

Derivatized antibodies may be used to alter the half-lives (e.g., serum half-lives) of parental antibodies in a mammal, particularly a human. Such alterations may result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present disclosure or fragments thereof in a mammal, preferably a human, results in a higher serum titer of said antibodies or antibody fragments in the mammal, and thus reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor.

Beltramello et al. (2010) previously reported the modification of neutralizing mAbs, due to their tendency to enhance dengue virus infection, by generating in which leucine residues at positions 1.3 and 1.2 of CH2 domain (according to the IMGT unique numbering for C-domain) were substituted with alanine residues. This modification, also known as “LALA” mutation, abolishes antibody binding to FcγRI, FcγRII and FcγRIIIa, as described by Hessell et al. (2007). The variant and unmodified recombinant mAbs were compared for their capacity to neutralize and enhance infection by the four dengue virus serotypes. LALA variants retained the same neutralizing activity as unmodified mAb but were completely devoid of enhancing activity. LALA mutations of this nature are therefore contemplated in the context of the presently disclosed antibodies.

Altered Glycosylation. A particular embodiment of the present disclosure is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan may be covalently attached to the heavy chain constant region.

Another embodiment of the present disclosure comprises a mAb with a novel Fc glycosylation pattern. The isolated monoclonal antibody, or antigen binding fragment thereof, is present in a substantially homogenous composition represented by the GNGN or G1/G2 glycoform. Fc glycosylation plays a significant role in anti-viral and anti-cancer properties of therapeutic mAbs. The disclosure is in line with a recent study that shows increased anti-lentivirus cell-mediated viral inhibition of a fucose free anti-HIV mAb in vitro. This embodiment of the present disclosure with homogenous glycans lacking a core fucose, showed increased protection against specific viruses by a factor greater than two-fold. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. In one embodiment of the present disclosure, the antibody dissociates from Fc gamma RI with a Kd of 1×10⁻⁸ M or less and from Fc gamma RIII with a Kd of 1×10⁻⁷ M or less.

Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to A1a can remove one of the glycosylation sites.

In certain embodiments, the antibody is expressed in cells that express beta (1,4)-N-acetylglucosaminyltransferase III (GnT III), such that GnT III adds GlcNAc to the IL-23p19 antibody. Methods for producing antibodies in such a fashion are provided in WO/9954342, WO/03011878, patent publication 20030003097A1, and Umana et al., Nature Biotechnology, 17:176-180, February 1999. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express recombinant monoclonal antibodies.

Elimination of monoclonal antibody protein sequence liabilities. It is possible to engineer the antibody variable gene sequences obtained from human B cells to enhance their manufacturability and safety. Potential protein sequence liabilities can be identified by searching for sequence motifs associated with sites containing:
1) Unpaired Cys residues,
2) N-linked glycosylation,
3) Asn deamidation,
4) Asp isomerization,
5) SYE truncation,
6) Met oxidation,
7) Trp oxidation,
8) N-terminal glutamate,
9) Integrin binding,
10) CD11c/CD18 binding, or
11) Fragmentation
Such motifs can be eliminated by altering the synthetic gene for the cDNA encoding recombinant antibodies.

Protein engineering efforts in the field of development of therapeutic antibodies clearly reveal that certain sequences or residues are associated with solubility differences (Fernandez-Escamilla et al., Nature Biotech., 22 (10), 1302-1306, 2004; Chennamsetty et al., PNAS, 106 (29), 11937-11942, 2009; Voynov et al., Biocon. Chem., 21 (2), 385-392, 2010) Evidence from solubility-altering mutations in the literature indicate that some hydrophilic residues such as aspartic acid, glutamic acid, and serine contribute significantly more favorably to protein solubility than other hydrophilic residues, such as asparagine, glutamine, threonine, lysine, and arginine.

Stability. Antibodies can be engineered for enhanced biophysical properties. One can use elevated temperature to unfold antibodies to determine relative stability, using average apparent melting temperatures. Differential Scanning Calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG1, IgG2, IgG3, and IgG4 subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95 °C and a heating rate of 1 °C/min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

Solubility. One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366:449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Autoreactivity. Generally, it is thought that autoreactive clones should be eliminated during ontogeny by negative selection, however it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

Preferred residues (“Human Likeness”). B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

### D. Single Chain Antibodies

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix-and turn-promoting amino acid residues such as alanine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338 describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

### E. Multispecific Antibodies

In certain embodiments, antibodies of the present disclosure are bispecific or multispecific. Bispecific antibodies are antibodies that have binding specificities for at least two different epitopes. Exemplary bispecific antibodies may bind to two different epitopes of a single antigen. Other such antibodies may combine a first antigen binding site with a binding site for a second antigen. Alternatively, an anti-pathogen arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess a pathogen-binding arm and an arm that binds the cytotoxic agent (e.g., saporin, anti-interferon-α, vinca alkaloid, ricin A chain, methotrexate or radioactive isotope hapten). Bispecific antibodies can be prepared as full-length antibodies or antibody fragments (e.g., F(ab′)₂ bispecific antibodies). WO 96/16673 describes a bispecific anti-ErbB2/anti-Fc gamma RIII antibody and U.S. Pat. No. 5,837,234 discloses a bispecific anti-ErbB2/anti-Fc gamma RI antibody. A bispecific anti-ErbB2/Fc alpha antibody is shown in WO98/02463. U.S. Pat. No. 5,821,337 teaches a bispecific anti-ErbB2/anti-CD3 antibody.

Methods for making bispecific antibodies are known in the art. Traditional production of full-length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

In a particular embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210(1986 ).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175:217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998). doi:10.1038/nbt0798-677pmid:9661204). For example, bispecific antibodies have been produced using leucine zippers (Kostelny et al., J. Immunol., 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368(1994 ).

In a particular embodiment, a bispecific or multispecific antibody may be formed as a DOCK-AND-LOCK™ (DNL™) complex (see, e.g., U.S. Pat. Nos. 7,521,056; 7,527,787; 7,534,866; 7.550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579:3264; Wong and Scott, Nat. Rev. Mol. Cell Biol. 2004; 5:959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147: 60, 1991; Xu et al., Science, 358(6359):85-90, 2017). A multivalent antibody may be internalized (and/or catabolized) faster than a bivalent antibody by a cell expressing an antigen to which the antibodies bind. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1)ₙ-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

Accordingly, in particular embodiments, an antibody comprised in the therapeutic agent comprises:
(a) a first Fab molecule which specifically binds to a first antigen
(b) a second Fab molecule which specifically binds to a second antigen, and wherein the variable domains VL and VH of the Fab light chain and the Fab heavy chain are replaced by each other,
wherein the first antigen is an activating T cell antigen and the second antigen is a target cell antigen, or the first antigen is a target cell antigen and the second antigen is an activating T cell antigen; and
wherein
i) in the constant domain CL of the first Fab molecule under a) the amino acid at position 124 is substituted by a positively charged amino acid (numbering according to Kabat), and wherein in the constant domain CH1 of the first Fab molecule under a) the amino acid at position 147 or the amino acid at position 213 is substituted by a negatively charged amino acid (numbering according to Kabat EU index); or
ii) in the constant domain CL of the second Fab molecule under b) the amino acid at position 124 is substituted by a positively charged amino acid (numbering according to Kabat), and wherein in the constant domain CH1 of the second Fab molecule under b) the amino acid at position 147 or the amino acid at position 213 is substituted by a negatively charged amino acid (numbering according to Kabat EU index).
The antibody may not comprise both modifications mentioned under i) and ii). The constant domains CL and CH1 of the second Fab molecule are not replaced by each other (i.e., remain unexchanged).

In another embodiment of the antibody, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 or the amino acid at position 213 is substituted independently by glutamic acid (E), or aspartic acid (D) (numbering according to Kabat EU index).

In a further embodiment, in the constant domain CL of the first Fab molecule under a) the amino acid at position 124 is substituted independently by lysine (K), arginine (R) or histidine (H) (numbering according to Kabat), and in the constant domain CH1 of the first Fab molecule under a) the amino acid at position 147 is substituted independently by glutamic acid (E), or aspartic acid (D) (numbering according to Kabat EU index).

In a particular embodiment, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)) and the amino acid at position 123 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In a more particular embodiment, in the constant domain CL of the first Fab molecule under a) the amino acid at position 124 is substituted by lysine (K) (numbering according to Kabat) and the amino acid at position 123 is substituted by lysine (K) or arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

In an even more particular embodiment, in the constant domain CL of the first Fab molecule under a) the amino acid at position 124 is substituted by lysine (K) (numbering according to Kabat) and the amino acid at position 123 is substituted by arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

### F. Purification

In certain embodiments, the antibodies of the present disclosure may be purified. The term “purified,” as used herein, is intended to refer to a composition, isolatable from other components, wherein the protein is purified to any degree relative to its naturally obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### III. Synthetic Receptors

Synthetic receptors comprise portions of natural receptors that are linked with other non-native elements to establish the ability to control cell behavior in response to environmental stimuli. A common form of synthetic receptor is to use N-terminal fusions with affinity domains, such as single chain variable fragments (scFvs), that bind ligands that activate the receptor. The intracellular receptor domain can be chosen from a variety of signal transduction domains of receptors to that endogenous signaling pathways. These systems are customizable in both the ligands to which the receptor responds and the pathways that are activated.

synNotch receptors are one type of artificially created receptor are used in synthetic biology applications. They are derived from naturally occurring Notch receptors, which is a family of transmembrane receptors participating in a wide range of cellular processes, for example embryogenesis, cardiovascular development, immunity and others. synNotch receptors enable a ligand-responsive transcriptional activation by a process analogous to the regular Notch receptors, enabling cellular programming at multiple levels simultaneously.

synNotch receptors have an extremely modular architecture and are comprised of three basic domains: an extracellular domain, a transmembrane domain and an intracellular domain. Of these three domains, only the architecture of the transmembrane domain that directs the proteolysis and release of the intracellular domain's transcription factor is retained from the original Notch receptors. The extracellular domain serves as an input sensing platform for various stimuli present on cellular surfaces of cells encountered by the bearer of synNotch receptor and the intracellular domain serves as a transcription factor responsible for functional output of the receptor. For the extracellular domain, the most used sensors are scFv, minibodies and nanobodies and are synthetic in their nature and for their intracellular domain transcription factors, a wide variety is utilized.

Like the Notch receptors, synNotch receptors have their activity triggered by stretching of the transmembrane domain, which activates its proteolytitic activity and transcription factor release in a dose dependent manner, which means that the transcriptional activation is modulated by the amount of ligand, and also by the amount of synNotch present (and by extension the strength of the promoter that drives its expression). The modality of those receptors can be governed by alterations made to this domain by changing the number of extracellular EGF-like domain repeats, which are part of the transmembrane domain. This is demonstrated by high basal activity for some versions of a given receptor, but addition of EGF-like domain repeats regulates this activity significantly and the receptor retains its activation potential upon introduction of the correct ligand. Their activity is also highly orthogonal with each other and also with the endogenous Notch receptors, thus minimizing their potential negative cross-activation effects.

synNotch receptors have a wide variety of potential applications, for example cell fate programming or multicellular structure programming. But their most notable potential in real life applications is in increasing the safety profile of CAR-T cell therapy by introduction of an AND gate, which means by increasing the number of ligands necessary for T cell activation. This is accomplished by a detection of a tumor-related input signal (for example CD19 in lymphoma), which activates the transcription of the CAR placed under promoter responsive to transcription factor release by synNotch, which then in turn senses another relevant tumor input and fully activates the CAR-T cell cytotoxic potential, thus reducing potential off-target activation mediated cytotoxicity.

SynNotch receptors represent an early example of artificial receptors based on ligand-regulated intramembrane proteolysis. Alternative receptors operating on a similar principle of previously been disclosed (Zhu et al., 2022), but are composed of modified juxtamembrane and intramembrane domains sensitive to ligand-dependent proteolytic cleavage. Such implementations of artificial recognition motifs are meant to be incorporated herein.

SNIPR, or Synthetic Intramembrane Proteolysis Receptor, was reported by Zhu et al., Cell, 185(8): 1431-1443.e16 (2022), incorporated by reference herein. These authors designed, assembled, and tested a large family of receptors that are compact in size, well-expressed, compatible with human and humanized synthetic TFs, readily tunable, and are both highly sensitive and specific to their target ligand. The term SNIPR can refer to synNotch, being that it falls within this class of artificial receptors, or the term can refer generally to artificial receptors with similar architectures (i.e., an extracellular affinity motif, juxtamembrane and transmembrane domains that are cleavable upon ligand binding by the extracellular affinity motif, and an intracellular transcription factor that regulates transgene expression from a cognate promoter). These molecules function robustly in SNIPR-chimeric antigen receptor (CAR) dual antigen-sensing circuits in vivo, a therapeutic strategy that enhances tumor specificity and therapeutic efficacy of engineered T cells for solid tumors. They also showed that rationally modified SNIPRs were able to achieve titratable production of therapeutic payloads, enabling spatially controlled and dosed delivery of therapeutic agents by cells at sites of disease.

### IV. Expression Constructs and Cell Engineering Permitting Transgene Expression

In some embodiments, genetic material is introduced into a cell, such as in vitro, ex vivo, or in vivo. For example, sequences that express anti-Aβ antibody sequences, detectable marker proteins and synthetic receptors SynNotch elements will all need to be introduced into a cell capable of expressing the same. In order express a protein, a nucleic acid must be transferred into the cell of interest under control of elements, such as promoters and enhancers, capable of supporting expression. This can be accomplished using an expression construct or vector containing such control elements, and also normally containing an origin of replication for reproduction of the vector.

By “expression construct,” “expression vector” or “expression cassette” is meant a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide to be delivered to a host cell, either in vitro or in vivo.

A “plasmid,” a common type of a vector, is an extra-chromosomal DNA molecule separate from the chromosomal DNA that is capable of replicating independently of the chromosomal DNA. In certain cases, it is circular and double-stranded.

An “origin of replication” (“ori”) or “replication origin” is a DNA sequence, e.g., in a lymphotrophic herpes virus, that when present in a plasmid in a cell is capable of maintaining linked sequences in the plasmid and/or a site at or near where DNA synthesis initiates. As an example, an ori for EBV includes FR sequences (20 imperfect copies of a 30 bp repeat), and preferably DS sequences; however, other sites in EBV bind EBNA-1, e.g., Rep* sequences can substitute for DS as an origin of replication (Kirshmaier and Sugden, 1998). Thus, a replication origin of EBV includes FR, DS or Rep* sequences or any functionally equivalent sequences through nucleic acid modifications or synthetic combination derived therefrom. For example, the present disclosure may also use genetically engineered replication origin of EBV, such as by insertion or mutation of individual elements, as specifically described in Lindner, et. al., 2008.

A “gene,” “polynucleotide,” “coding region,” “sequence,” “segment,” “fragment,” or “transgene” that “encodes” a particular protein, is a nucleic acid molecule that is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (IRES), enhancers, splice junctions, and the like, which collectively provide for the replication, transcription, post-transcriptional processing, and translation of a coding sequence in a recipient cell. Not all of these control elements need be present so long as the selected coding sequence is capable of being replicated, transcribed, and translated in an appropriate host cell.

The term “promoter” is used herein in its ordinary sense to refer to a nucleotide region comprising a DNA regulatory sequence, wherein the regulatory sequence is derived from a gene that is capable of binding RNA polymerase and initiating transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

By “enhancer” is meant a nucleic acid sequence that, when positioned proximate to a promoter, confers increased transcription activity relative to the transcription activity resulting from the promoter in the absence of the enhancer domain.

By “operably linked” or co-expressed” with reference to nucleic acid molecules is meant that two or more nucleic acid molecules (e. g., a nucleic acid molecule to be transcribed, a promoter, and an enhancer element) are connected in such a way as to permit transcription of the nucleic acid molecule. “Operably linked” or “co-expressed” with reference to peptide and/or polypeptide molecules means that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of heterologous molecules.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally occurring cells or artificially modified cells (e.g., fusion cells, genetically modified cells, etc.).

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPSCs”.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

In some embodiments, a vector comprises a regulatory element operably linked to an coding sequence. In some embodiments, a coding sequence is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in tum believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In some aspects, polynucleotide is introduced to the cell. The polynucleotide typically is provided in the form of an expression vector, such as a viral expression vector. In some aspects, the expression vector is a viral expression vector or a DNA plasmid expression vector. Alternatively, an mRNA is used. One of skill in the art would be well-equipped to construct a vector through standard recombinant techniques (see, for example, Sambrook et al., 2001 and Ausubel et al., 1996, both incorporated herein by reference). Vectors include but are not limited to, plasmids, cosmids, viruses (bacteriophage, animal viruses, and plant viruses), and artificial chromosomes (e.g., YACs), such as retroviral vectors (e.g., derived from Moloney murine leukemia virus vectors (MoMLV), MSCV, SFFV, MPSV, SNV etc), lentiviral vectors (e.g., derived from HIV-1, HIV-2, SIV, BIV, FIV, etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors.

In some embodiments, the polypeptides are synthesized in situ in the cell as a result of the introduction of polynucleotides encoding the polypeptides into a cell. Methods for introducing a polynucleotide construct into animal cells are known and include, as non-limiting examples stable transformation methods wherein the polynucleotide construct is integrated into the genome of the cell, transient transformation methods wherein the polynucleotide construct is not integrated into the genome of the cell, and virus mediated methods. In some embodiments, the polynucleotides may be introduced into the cell by for example, recombinant viral vectors (e.g., retroviruses, adenoviruses), liposome and the like. For example, in some aspects, transient transformation methods include microinjection, electroporation, or particle bombardment. In some embodiments, the polynucleotides may be included in vectors, more particularly plasmids or virus, in view of being expressed in the cells.

Transposase/transposon systems are another useful way of delivering nucleic acids. Examples include the Sleeping Beauty transposase/transposon system and piggyBac transposase/transposon system. Others with equivalent functions are also contemplated.

In some embodiments, delivery is via the use of RNA or DNA viral based systems for the delivery of nucleic acids. Viral vectors in some aspects may be administered directly to patients (in vivo) or they can be used to treat cells in vitro or ex vivo, and then administered to mice. Viral-based systems in some embodiments include retroviral, lentivirus, adenoviral, adeno-associated and herpes simplex virus vectors for gene transfer.

Viral vectors may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor-mediated endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present disclosure are described below.

Retroviruses have promise as gene delivery vectors due to their ability to integrate their genes into the host genome, transfer a large amount of foreign genetic material, infect a broad spectrum of species and cell types, and be packaged in special cell-lines (Miller, 1992).

In order to construct a retroviral vector, a nucleic acid is inserted into the viral genome in place of certain viral sequences to produce a virus that is replication-defective. In order to produce virions, a packaging cell line containing the gag, pol, and env genes—but without the LTR and packaging components—is constructed (Mann et al., 1983). When a recombinant plasmid containing a cDNA, together with the retroviral LTR and packaging sequences, is introduced into a special cell line (e.g., by calcium phosphate precipitation), the packaging sequence allows the RNA transcript of the recombinant plasmid to be packaged into viral particles, which are then secreted into the culture medium (Nicolas and Rubenstein, 1988; Temin, 1986; Mann et al., 1983). The medium containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

In a particular embodiment, the inventors contemplate the use of adeno-associated virus (AAV), a small nonpathogenic virus of the Parvoviridae family. To date, numerous serologically distinct AAVs have been identified, and more than a dozen have been isolated from humans or primates. AAV is distinct from other members of this family by its dependence upon a helper virus for replication.

AAV genomes can exist in an extrachromosomal state without integrating into host cellular genomes; possess a broad host range; transduce both dividing and non-dividing cells in vitro and in vivo and maintain high levels of expression of the transduced genes. AAV viral particles are heat stable; resistant to solvents, detergents, changes in pH, and temperature; and can be column purified and/or concentrated on CsCl gradients or by other means. The AAV genome comprises a single-stranded deoxyribonucleic acid (ssDNA), either positive-or negative-sensed. The approximately 4.7 kb genome of AAV consists of one segment of single stranded DNA of either plus or minus polarity. The ends of the genome are short-inverted terminal repeats (ITRs) that can fold into hairpin structures and serve as the origin of viral DNA replication.

An AAV “genome” refers to a recombinant nucleic acid sequence that is ultimately packaged or encapsulated to form an AAV particle. An AAV particle often comprises an AAV genome packaged with AAV capsid proteins. In cases where recombinant plasmids are used to construct or manufacture recombinant vectors, the AAV vector genome does not include the portion of the “plasmid” that does not correspond to the vector genome sequence of the recombinant plasmid. This non-vector genome portion of the recombinant plasmid is referred to as the “plasmid backbone,” which is important for cloning and amplification of the plasmid, a process that is needed for plasmid propagation and production but is not itself packaged or encapsulated into viral particles. Thus, an AAV vector “genome” refers to nucleic acid that is packaged or encapsulated by AAV capsid proteins.

The AAV virion (particle) is a non-enveloped, icosahedral particle approximately 25 nm in diameter that comprises an AAV capsid. The AAV particle comprises an icosahedral symmetry comprised of three related capsid proteins, VP1, VP2 and VP3, which interact together to form the capsid. The genomes of most native AAVs often contain two open reading frames (ORFs), sometimes referred to as a left ORF and a right ORF. The right ORF often encodes the capsid proteins VP1, VP2, and VP3. These proteins are often found in a ratio of 1:1:10 respectively, but may be in varied ratios, and are all derived from the right-hand ORF. The VP1, VP2 and VP3 capsid proteins differ from each other by the use of alternative splicing and an unusual start codon. Deletion analysis has shown that removal or alteration of VP1 which is translated from an alternatively spliced message results in a reduced yield of infectious particles. Mutations within the VP3 coding region result in the failure to produce any single-stranded progeny DNA or infectious particles. In certain embodiments, the genome of an AAV particle encodes one, two or all three VP1, VP2 and VP3 polypeptides. In addition to natural capsids of wild-type AAVs, use of engineered capsids is contemplated herein for production of recombinant AAV.

The left ORF often encodes the non-structural Rep proteins, Rep 40, Rep 52, Rep 68 and Rep 78, which are involved in regulation of replication and transcription in addition to the production of single-stranded progeny genomes. Two of the Rep proteins have been associated with the preferential integration of AAV genomes into a region of the q arm of human chromosome 19. Rep68/78 have been shown to possess NTP binding activity as well as DNA and RNA helicase activities. Some Rep proteins possess a nuclear localization signal as well as several potential phosphorylation sites. In certain embodiments the genome of an AAV (e.g., an rAAV) encodes some or all of the Rep proteins. In certain embodiments the genome of an AAV (e.g., an rAAV) does not encode the Rep proteins. In certain embodiments one or more of the Rep proteins can be delivered in trans and are therefore not included in an AAV particle comprising a nucleic acid encoding a polypeptide.

The ends of the AAV genome comprise short-inverted terminal repeats (ITR) which have the potential to fold into T-shaped hairpin structures that serve as the origin of viral DNA replication. Accordingly, the genome of an AAV comprises one or more (e.g., a pair of) ITR sequences that flank a single stranded viral DNA genome. The ITR sequences often have a length of about 145 bases each. Within the ITR region, two elements have been described which are believed to be central to the function of the ITR, a GAGC repeat motif and the terminal resolution site (trs). The repeat motif has been shown to bind Rep when the ITR is in either a linear or hairpin conformation. This binding is thought to position Rep68/78 for cleavage at the trs which occurs in a site-and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

In some embodiments, viral and non-viral based gene transfer methods can be used to introduce nucleic acids in mammalian cells or target tissues. Such methods can be used to administer nucleic acids encoding components of a CRISPR to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, 1992; Nabel & Feigner, 1993; Mitani & Caskey, 1993; Dillon, 1993; Miller, 1992; Van Brunt, 1988; Vigne, 1995; Kremer & Perricaudet, 1995; Haddada et al., 1995 and Yu et al., 1994.

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in, e.g., U.S. Pat. Nos. 5,049,386, 4,946,787, and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91117424; WO 91116024. Delivery can be to cells (e.g., in vitro or ex vivo administration) or target tissues (e.g., in vivo administration).

In certain embodiments of the present disclosure, a nucleic acid is introduced into an organelle, a cell, a tissue or an organism via electroporation. Electroporation involves the exposure of a suspension of cells and DNA to a high-voltage electric discharge. Recipient cells can be made more susceptible to transformation by mechanical wounding. Also, the amount of vectors used may vary upon the nature of the cells used, for example, about 5 to about 20 μg vector DNA per 1 to 10 million of cells may be contemplated.

Expression cassettes included in vectors useful in the disclosure preferably contain (in a 5′-to-3′ direction) a eukaryotic transcriptional promoter operably linked to a protein-coding sequence, splice signals including intervening sequences, and a transcriptional termination/polyadenylation sequence.

The expression constructs provided herein comprise promoter to drive expression of the programming genes. A promoter generally comprises a sequence that functions to position the start site for RNA synthesis. The best-known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCRTM, in connection with the compositions disclosed herein (see U.S. Pat. Nos. 4,683,202 and 5,928,906, each incorporated herein by reference). Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al., 1989, incorporated herein by reference). The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters (e.g., CAG promoter, Rous Sarcoma Virus (RSV) early promoters; eukaryotic cell promoters, such as, e.g., beta actin promoter (Ng, 1989; Quitsche et al., 1989), GADPH promoter (Alexander et al., 1988, Ercolani et al., 1988), beta globulin promoter, metallothionein promoter (Karin et al., 1989; Richards et al., 1984); and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007).

Tissue-specific transgene expression, especially for reporter gene expression in hematopoietic cells and precursors of hematopoietic cells derived from programming, may be desirable as a way to identify derived hematopoietic cells and precursors. To increase both specificity and activity, the use of cis-acting regulatory elements has been contemplated. For example, an astrocyte cell-specific promoter (i.e., GfaBC₁D promoter) may be used.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

In particular aspects, the promoter is an inducible promoter. The activity of inducible promoters may be induced by the presence or absence of biotic or abiotic factors. Inducible promoters are a very powerful tool in genetic engineering because the expression of genes operably linked to them can be turned on or off at certain stages of development of an organism or in a particular tissue. For example, Tet-On and Tet-Off inducible gene expression systems based on the essential regulatory components of the E. coli tetracycline-resistance operon may be used. Once established in the starting cells, the inducer doxycycline (Dox, a tetracycline derivative) could control the expression system in a dose-dependent manner, allowing the precise modulation of the expression levels of programming genes. In exemplary embodiments, the inducible promoter is an rtTET-inducible Tight promoter (pTight). Thus, the pTight promoter could be used to induce expression of the multi-lineage programming genes such as ETV2, GATA2 and HOXA9 for a period of time sufficient to allow programming of the PSCs to hematopoietic precursor cells, and the expression could subsequently be turned off. The pTight promoter could also be a bi-directional promoter. Another non-limiting example is the UAS promoter used with the Gal4 system. Human lineage-specific promoters, such as a PAX6 response element, may also be deployed as inducible promoters along with a cognate transcriptional activator, e.g., PAX6-VP64, in otherwise lineage-negative cells.

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the disclosure, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference). As an alternative to IRES elements, skipping peptide sequences (e.g., E2A, F2A, P2A, T2A) can be used for polycistronic transgene expression (Szymczak-Workman et al., 2007).

### V. Transgenes

A variety of different transgenes may be introduced into the engineered cells described above. For in vitro research or in vivo diagnostics, a detectable marker may be utilized. Examples include Green Fluorescent Protein, mCherry, Blue fluorescent protein, infrared fluorescent proteins, mCardinal, firefly luciferase, nanoluciferase, BRET reporters (e.g., lumiflour), FRET reporters, secreted alkaline phosphatase, gaussian luciferase, Renilla luciferase, or MRI reporters (OATP1B3 transporter).

The transgene can also be a therapeutic protein. Examples include brain-derived neurotrophic factor (BDNF), presenilin, anti-inflammatory proteins such as interleukin-10, transforming growth factor beta 1 (TGFβ1), fibroblast growth factor (FGF), native inhibitors of interleukin-1, (e.g., IL1Ra) interleukin-6, or tumor necrosis factor (e.g., sTNFR1) or biologic neutralizing factors (canakinumab, ziltivekimab, infliximab, adalimumab); inhibitors of complement or a cascade factor; amyloid clearing factors (neprilysin, anti-Aβ antibody); and anti-tau factors (e.g., bepranemab).

The transgene can also be a protein that can promote an Alzheimer's Disease phenotype, such as for the creation of disease models. Examples include variants of TREM2, CD33, APOE, SERPINA3, CLU, BIN1, SORL1, ABCA7, ADAM10, APP, PSEN1, or IGHG3.

### VI. Formulation and Administration

The present disclosure provides pharmaceutical compositions comprising expression vectors or cells as described here. Such compositions comprise an effective amount of and expression vector or cell in a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, or emulsions. Examples of suitable pharmaceutical agents are described in “Remington's Pharmaceutical Sciences.” Such compositions will contain a prophylactically or therapeutically effective amount of the compositions, preferably in purified form, together with a suitable amount of carrier so as to provide the form for proper administration to the patient. The formulation should suit the mode of administration, which can be intracranial, intravenous or intraarterial.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### VII. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

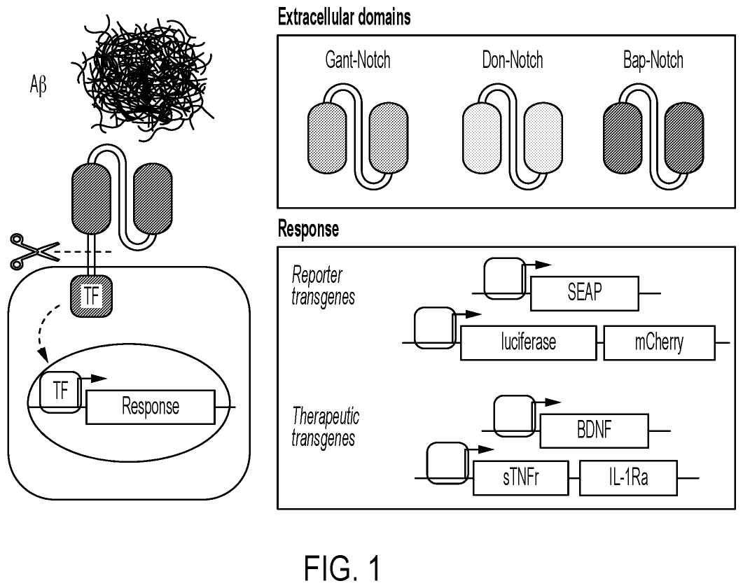

The inventors have built synthetic Notch (synNotch) (Roybal et al., 2016a; Morsut et al., 2016) receptors that allow cells to respond to amyloid-b (Aβ) oligomers via customized transgene expression programs. SynNotch is based on the native juxtacrine Notch signaling channel (Morsut et al., 2016; Roybal et al., 2016b), which requires mechanical strain generated by immobilized ligand for receptor activation (FIG. 1) (Luca et al., 2017). Free, monomeric soluble ligand does not activate this receptor. As such, synNotch activation is exquisitely selective to Aβ oligomers or immobilized plaques as opposed to soluble monomers. By exchanging Notch's (1) extracellular domain with alternative recognition motifs (e.g., single-chain variable fragments [scFvs]) and (2) intracellular domain with a synthetic transcription factor, the inventors create synNotch receptors that produce user-specified sense/response behaviors. For simplicity, a transgene whose expression is governed by synNotch signaling is referred to as “payload.”

By programming the recognition domain of synNotch with scFvs derived from monoclonal antibodies (mAbs) against Aβ, the inventors can produce customized responses to Aβ aggregates, driving cells to implement putative neurodegenerative or protective gene expression programs. Further, by choosing mAbs with purported specificity for particular Aβ species such as soluble oligomers, fibrils, or plaques, or parenchymal versus vascular amyloid, the inventors hypothesize that they can tailor targeted behaviors based on discrimination between these various forms of Aβ.

Thus, the inventors produced a panel of three Aβ-sensitive synNotch receptors, where each receptor is designed with an scFv recognition motif derived from the variable domains of different commercially available mAbs. The mAbs are known as Bapineuzumab (selective for fibrillar Aβ or higher ordered aggregates), Donanemab (selective for Aβ plaques), and Gantenerumab (selective for fibrillar Aβ or higher ordered aggregates). They refer to these receptors as Bap-Notch, Don-Notch, or Gant-Notch, respectively.

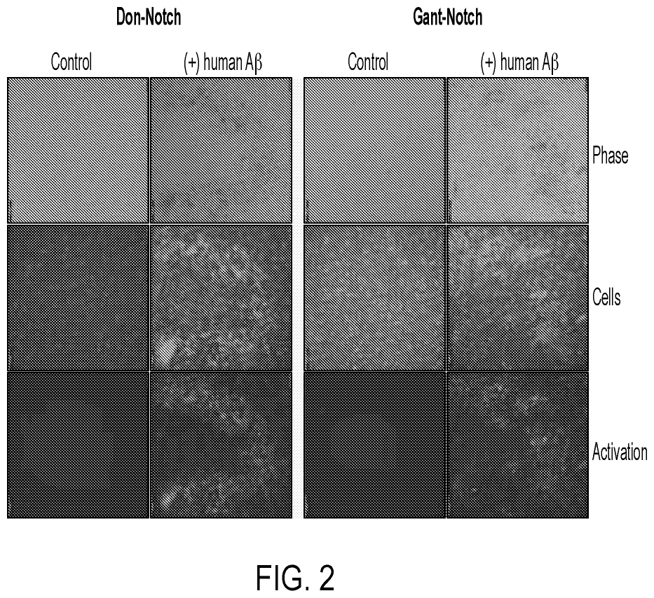

In one experiment, the inventors programmed murine L929 fibroblasts with one of three Aβ-sensitive synNotch variants: Gant-Notch, Don-Notch, Bap-Notch. In this experiment, all synNotch-programmed cells express blue fluorescent protein (BFP), and upon activation of synNotch signaling, cells also express mCherry fluorescent protein as well as firefly luciferase payloads. The inventors used Aβ extracted from vasculature of human brain donors validate that these receptors can detect human brain-derived amyloid. These amyloid “seeds” range in size from 0.5-7 microns and are free of tau contaminants, as assessed by western blot. They adsorbed a small volume (10 ml) of the suspension of human brain-derived Aβ seeds onto a cell culture dish and then plated each of the three synNotch fibroblast cell lines onto either the Aβ-treated surfaces or control, untreated surfaces. The results show that Don-Notch and Gant-Notch, receptors composed of variable domains from mAbs that recognize vascular and parenchymal amyloid, are able to activate mCherry expression upon treatment with the vascular-derived Aβ seeds (FIG. 2). Careful examination of the phase contrast micrographs shows increased material density in regions of the Aβ-treated cell culture surfaces, which likely corresponds to the location of the adsorbed Aβ seeds. These regions correlate to those in which the highest mCherry expression is observed by fluorescence microscopy. These results show that at least two of the receptors drive transgene expression in response to human brain-derived Aβ.

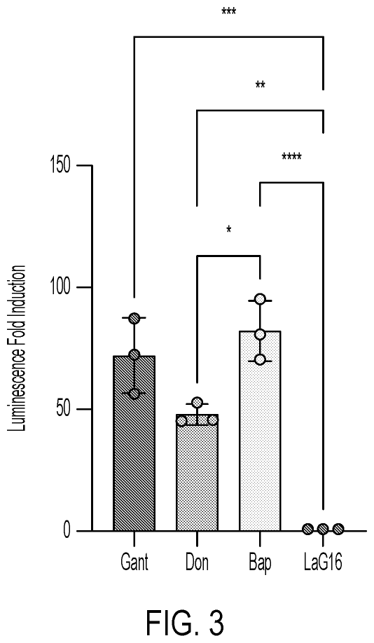

The inventors then transitioned to characterize functionality of Bap-Notch, Don-Notch, and Gant-Notch. Due to the properties of the mAbs used to generate these receptors, cells expressing any of these synNotch variants should be capable of detecting both Aβ₄₂ or Aβ₄₀. Thus, they compared the level of synNotch-driven luciferase expression by performing a Brightglo luminescence assay on L929 cells programmed with these three synNotch receptors. Luciferase enzyme activity from cells cultured in the presence of Aβ was normalized to expression levels when cells were cultured in the absence of Aβ, and results are expressed as fold-change luminescence relative to the Aβ-free condition. To control for generic synNotch performance, the inventors also included a GFP-sensitive synNotch, LaG16-Notch, in these experiments. Further, to control for differences in cell line generation, all cells were sorted via fluorescence activated cell sorting on the basis of synNotch receptor elements. They coupled C-terminally biotinylated Aβ42 peptides (Anaspec) to streptavidin-treated dishes (treatment: applied 40 ml of a solution of 50 mg/ml peptide to each well). Bapineuzumab, Donanemab, and Gantenerumab recognize epitopes on the N-terminal or central regions of Aβ, suggesting the C-terminal biotinylation should not interfere with receptor binding. They found that the panel of three Aβ-sensitive synNotch receptors display varied sensitivities to Aβ, while the GFP-sensitive LaG16-Notch does not respond to Aβ. (FIG. 3). In particular, Bap-Notch and Gant-Notch cells upregulated transgene expression by >70-fold in response to Aβ42, whereas Don-Notch achieved potent but reduced activation (~49-fold).

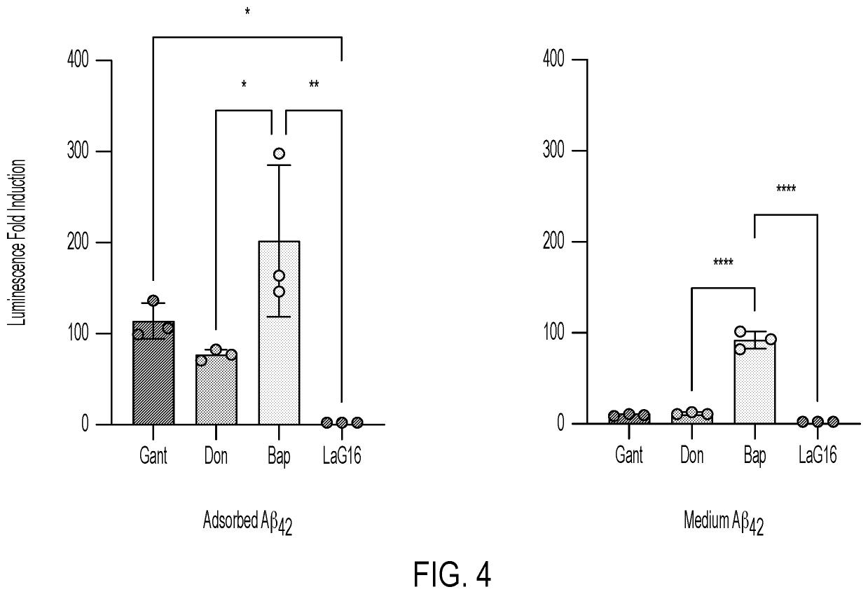

To account for the possible influence of C-terminal biotinylation, the inventors also performed experiments in which solutions (40 ul of a solution of 50 μg/ml peptide) of non-biotinylated Aβ42 were applied to culture vessels either as absorbates or as culture medium supplements. They again observed marked activation of the Bap-Notch receptor after either adsorption (~200-fold) or medium supplementation (92-fold) with Aβ42, while activation levels of Don-Notch and Gant-Notch (~75-110-fold, respectively) were observable after adsorption of Aβ42 but were limited after medium supplementation (~10-fold induction) (FIG. 4). These results suggest that Aβ-sensitive synNotch receptors such as Gant-Notch and Don-Notch are sensitive to substrate-mediated anchoring of Aβ42 oligomers, whereas Bap-Notch receptors are able to more effectively capture and immobilize oligomers/aggregates to enable the conformational changes required for receptor activation. This could be, in part, due to the enhanced affinity of Bapineuzumab to fibrillar Aβ42 as compared to Gantenerumab (0.32 nM versus 29 nM, respectively), and the fact that Donanemab is purportedly sensitive to N-terminal truncated, pyr3-42Aβ rather than Aβ42.

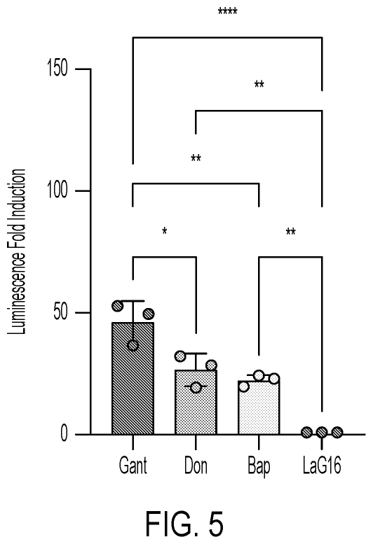

In parallel with the experiments that probed receptor activation levels from biotinylated, passively adsorbed, and medium supplemented Aβ42, the inventors also tested sensitivity of receptor variants to identical forms of Aβ40 treatment. Aβ42 is less soluble than Aβ40, and therefore Aβ40 is less likely to form aggregates. In agreement with this, synNotch transgene induction by Aβ40 treatment was diminished for all receptor variants as compared to Aβ42 treatment. Further, the trend of receptor responsiveness differed according to Aβ40 versus Aβ42 treatment, in that Gant-Notch (46-fold), rather than Bap-Notch, was more potently activated by Aβ40, while Bap-Notch and Don-Notch were activated to comparable levels (22-fold and 26-fold, respectively) (FIG. 5). Collectively, results indicate that the panel of artificial Aβ receptors effectively regulate transgene expression in engineered cells, the receptors are differentially sensitive to synthetic Aβ40, Aβ42, and brain-derived Aβ seeds, and the magnitude of cellular response to Aβ is dependent on the selected receptor variant.

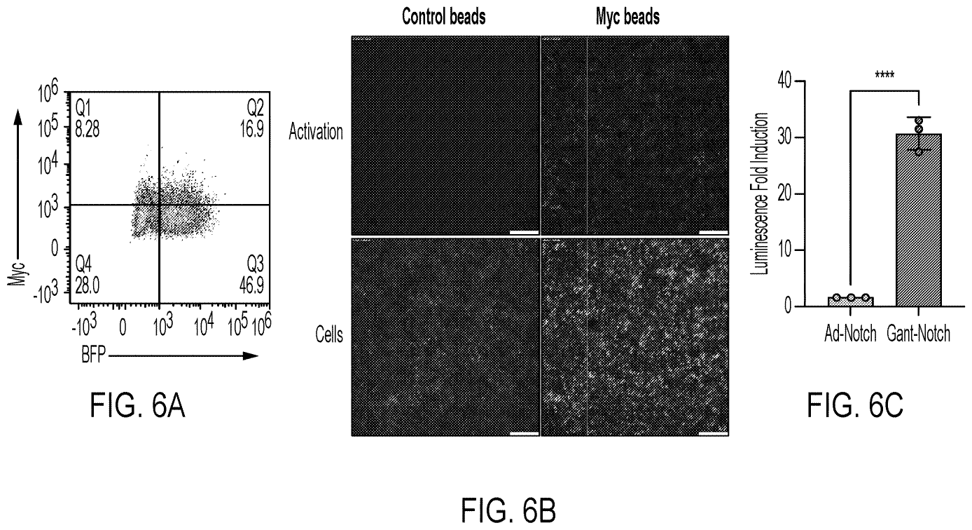

In addition to the receptors described above, the inventors constructed a synNotch receptor with an extracellular recognition domain constructed from the mAb aducanumab (Ad-Notch). This receptor was also expressed in L929 mouse fibroblasts, indicated by expression of the constitutive BFP protein and the N-terminal c-myc epitope tag present on the receptor (FIG. 6A). As with Gant-Notch, Don-Notch, and Bap-Notch, the Ad-Notch cells were sorted via fluorescence activated cell sorting on the basis of these synNotch receptor elements (i.e., for the double positive BFP, Myc population). After sorting, they validated that the Ad-Notch receptor is functional in cells by artificially activating the receptor using an anti-myc antibody conjugated to a 1 μm magnetic bead. These beads serve as surrogate synNotch ligands that enable uniform activation independent of target ligand for which the receptor is built (e.g., Aβ). Using this mode of activation, all three receptors induced potent levels of synNotch-driven mCherry expression (FIG. 6B). However, the Ad-Notch receptor was unable to recognize Aβ when cells were plated on biotinylated synthetic Aβ₄₂ (~1-fold induction, compared to >30 fold induction with Gant-Notch cells in the same experiment (FIG. 6 °C). Not shown is a similar result using human brain-derived amyloid seeds, in which Ad-Notch failed to produce mCherry transgene expression as measured by fluorescence microscopy, while Gant-Notch and Don-Notch did. These data suggest that Ad-Notch, though expressed on the cell surface and competent to generate signaling upon myc-reactive bead stimulation, is unresponsive to Aβ. This is distinct from reported results from studies of aducanumab-based chimeric antigen receptors deployed in macrophages. Due to its inability to recognize Aβ, the inventors removed Ad-Notch in subsequent characterization experiments.

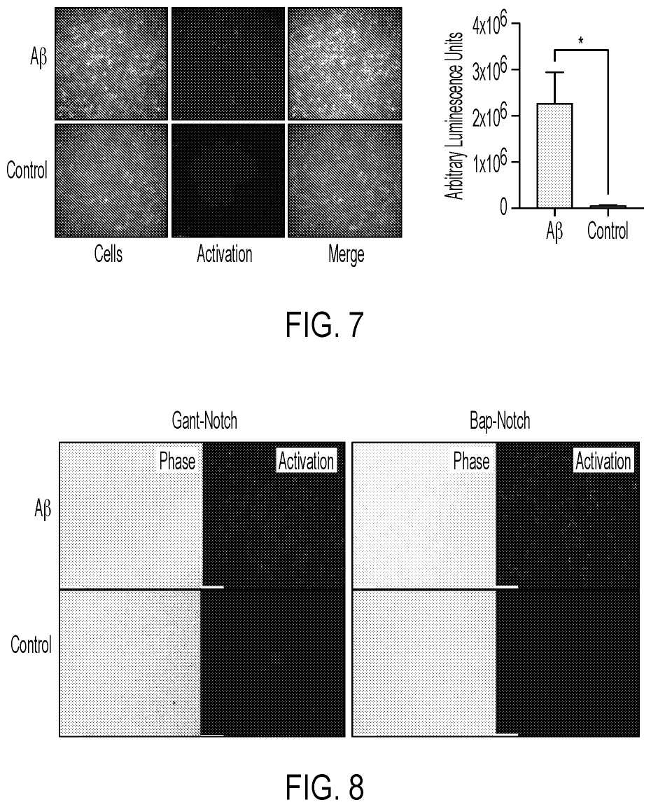

To determine whether Aβ-sensitive synNotch receptors can regulate behaviors of cells other than fibroblasts, the inventors ported the platform into other cell types. Mesenchymal stem cells (MSCs) have been clinically investigated as a cell-based Alzheimer's disease therapy to ameliorate inflammation and secrete neurotrophic factors that may mitigate disease. Thus, they transduced primary murine MSCs with the Gant-Notch receptor platform and exposed the cells to synthetic Aβ42. Microscopy and luminescence results indicate potent upregulation of mCherry and luciferase transgenes, respectively, from this artificial signaling platform (FIG. 7).

Pluripotent stem cells are also investigated as a source for cell-based therapies for Alzheimer's disease, as these cells can be used to generate neural stem cells as well as astrocytes and microglia, all of which are perturbed in AD. Thus, the inventors engineered pluripotent H9 human embryonic stem cells with Gant-Notch and Bap-Notch receptors and exposed them to Aβ42 peptide. They observed marked, Aβ-dependent upregulation of the synNotch transgene mCherry in these cells (FIG. 8), indicating that Aβ-synNotch variants can govern gene expression programs in pluripotent stem cells.

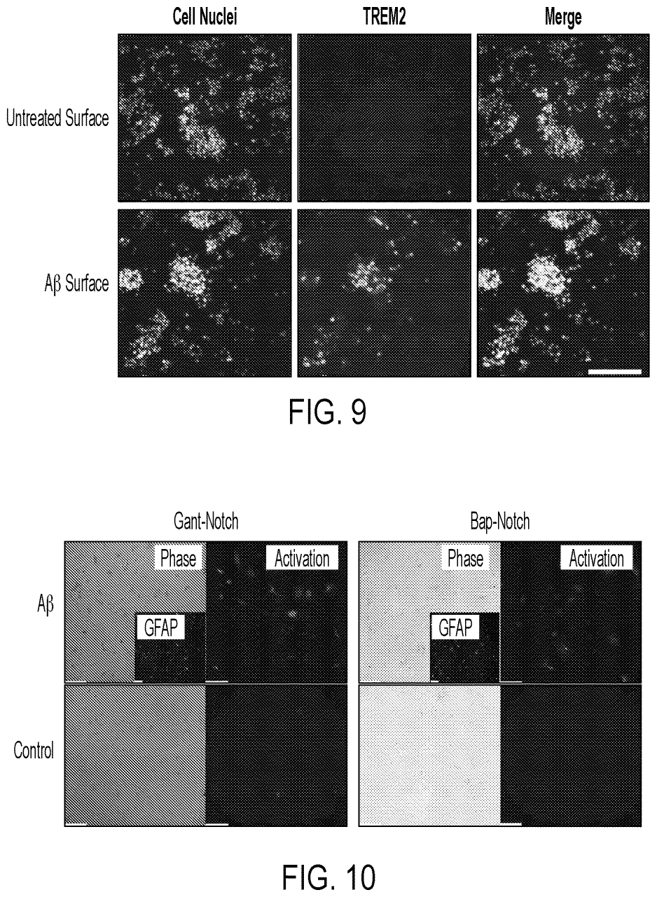

The inventors then shifted their focus to exploit this synthetic mode of signaling to determine whether they could regulate expression of genes involved in AD biology via Aβ-synNotch receptors. A major focus of recent AD investigations has been on the TREM2 signaling axis. In the brain, TREM2 is uniquely expressed by microglia, and the human TREM2 R47H variant increases susceptibility to AD by 3-fold (Guerreiro et al., 2013; Jonsson et al., 2013), second in known genetic susceptibility only to APOE variation. TREM2 protein interacts with other known AD-related variants since it serves as a surface receptor for both ApoE and Aβ (Yeh et al., 2016; Atagi et al., 2015; Zhao et al., 2018), though the precise ligand responsible for activating TREM2 is not clearly established (Ulrich et al., 2017). The role of TREM2 in amyloidosis is poorly defined. Transgenic animal investigations indicate that TREM2 plays a stage-specific and model-dependent role in disease progression (Gratuze et al., 2018). In in vivo models characterized by amyloidosis, TREM2 knockout has been reported to reduce disease burden early in the appearance of pathology, while TREM2 deficiency increases amyloid burden in late stages of disease (Jay et al., 2017; Wang et al., 2015; Bailey et al., 2015). Thus, the influence of TREM2 on microglia function and neurodegeneration remains a primary question in the field. One possible way to resolve questions pertaining to the directionality of amyloid burden and TREM2 function is to selectively suppress and/or activate expression of TREM2 in an Aβ-dependent manner. The experiments show that the inventors can induce mouse fibroblasts, a non-myeloid cell type, to express TREM2 as a payload transgene in response to Aβ-activation of synNotch (FIG. 9). This experiment illustrates the potential of using Aβ-sensitive synNotch receptors to reconstitute expression of putatively neuroprotective or neurodegenerative genes in order to investigate cause/effect relations in the progression of AD.

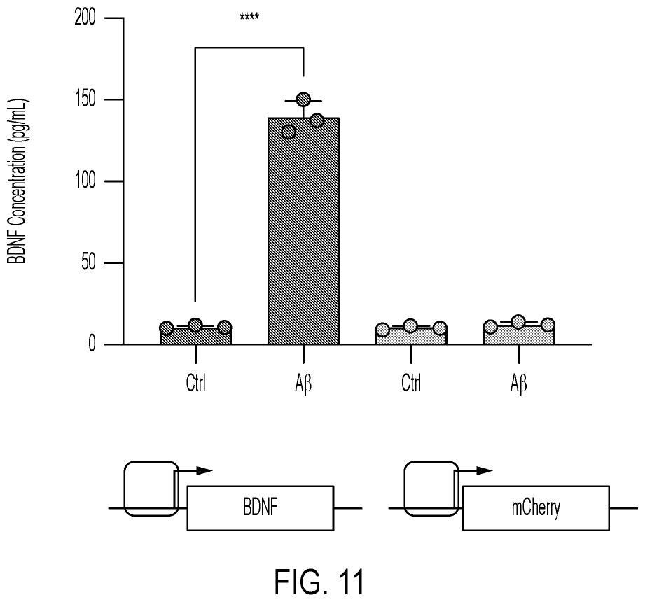

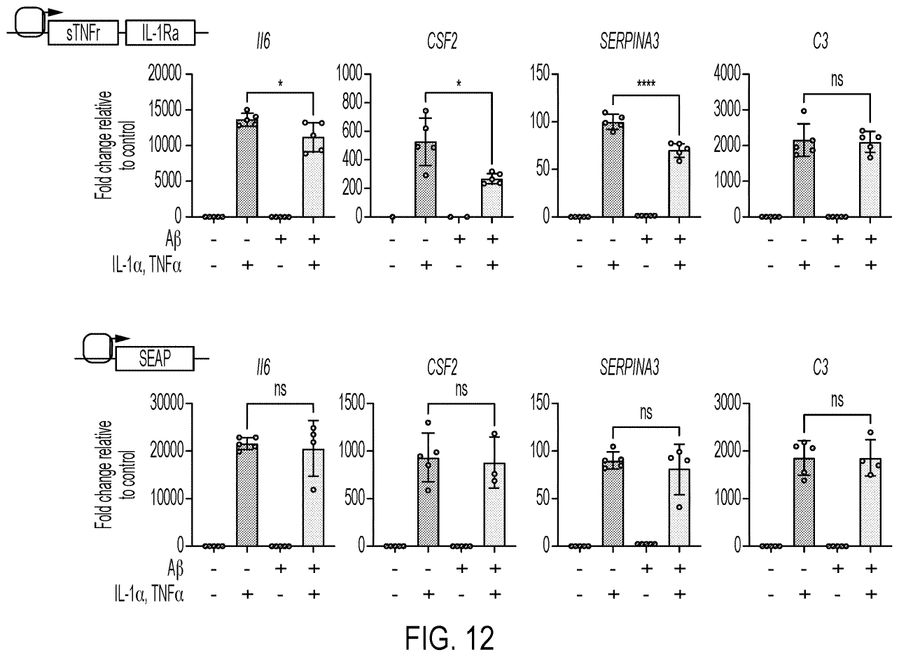

Next, the inventors took astrocytes derived from the induced pluripotent stem cell line CC3 and engineered them to express either mCherry or brain-derived neurotrophic factor (BDNF) under the control of synNotch signaling. BDNF is an investigational AD therapy, that has been shown to reduce neuronal death and improve synapse formation as well as hippocampal-dependent learning and memory in non-human primate models (Nagahara et al., 2013; Nagahara et al., 2009; Nagahara et al., 2018; Nagahara & Tuszynski 2011; Blurton-Jones et al., 2009). However, broadly distributed or ectopically expressed BDNF induces adverse effects including weight loss, sensory disturbances, and inappropriate cellular migratory patterns (Nagahara & Tuszynski 2011). This motivates the need to restrict BDNF expression to regions of the AD brain that exhibit a biochemical hallmark of disease-specific pathology, such as Aβ accumulation (Knopman et al., 2021; Butterfield& Halliwell, 2019; Baik et al., 2019; Tönnies & Trushina, 2017). When the inventors treated synNotch-programmed astrocytes with human brain-derived Aβ, they observed expression of the synNotch reporter mCherry in engineered astrocytes (FIG. 10). Remarkably, inducible BDNF protein production was detected in astrocytes engineered with the BDNF payload (FIG. 11). Enhanced, Aβ-activated BDNF production was not observed in synNotch-mCherry astrocytes, indicating that Aβ alone is not sufficient to cause the astrocytes to considerably upregulate BDNF production. Rather, only the combined influence of programming BDNF as the synNotch payload and culturing the cells in the presence of Aβ resulted in potent BDNF expression.

The inventors then programmed the synNotch astrocytes to produce antagonists to inflammatory cytokines TNF-α and IL-1α (soluble TNF receptor (sTNFr) and IL-1Ra, respectively). AD is characterized by chronic neuroinflammation, mediated in part by astrocytes (Knopman et al., 2021; Singh et al., 2022). In the context of brain injury or disease, activated microglia induce astrocytes to become reactive. Reactive astrocytes have been classified into 2 phenotypes: A1 and A2. A1 astrocytes are known to be neurotoxic and make up a large proportion of the astrocytes found in areas of neurodegeneration, including in AD. An A1 reactive astrocyte phenotype can be induced in vitro through addition of inflammatory cytokines TNFα, IL-1α, and C1q (Leng et al., 2022, Liddelow et al., 2017, Guttenplan et al., 2021, Guttikonda et al., 2021); additionally, the inventors have shown that TNFα and IL-1α alone is sufficient to induce a reactive astrocyte phenotype (Kim et al., 2022). Addition of TNFα and IL-1α to the SynNotch astrocytes results in an upregulation in the expression of A1 astrocyte reactive genes Il6, CSF2, SERPINA3, and C3 (FIG. 11). When SynNotch astrocytes produce sTNFr and IL-1Ra in response to Aβ exposure, there is a significant decrease in the expression of several of the genes induced by cytokine treatment (Il6, CSF2, SERPINA3), indicating that the synNotch-driven sTNFr and IL-1Ra are able to antagonize the inflammatory cytokines and attenuate the reactive astrocyte phenotype. When Bap-Notch astrocytes are instead engineered to express a synNotch-driven control reporter transgene secreted embryonic alkaline phosphatase (SEAP), there is no difference in the expression of reactive astrocyte genes when cells are cultured on Aβ.

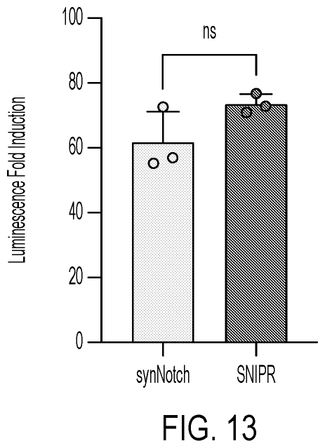

Finally, the inventors sought to confirm the ability of the Aβ recognition domain derived from bapineuzumab to function in the SNIPR (Synthetic Intramembrane Proteolysis Receptor) platform; SNIPR is a further engineering of the SynNotch platform that is constructed from truncated NOTCH1 juxtamembrane and transmembrane domains (Zhu et al) and whose smaller size is more amenable to delivery methods with smaller packaging limits (i.e., AAV). The inventors constructed a SNIPR receptor with a bapineuzumab extracellular domain (Bap-SNIPR). The Bap-SNIPR was expressed in mouse mesenchymal stem cells (mMSCs), and the cells were plated on synthetic Aβ₄₂. Bap-SNIPR cells displayed comparable Aβ-dependent expression of a luciferase transgene as compared to the mMSCs programmed with a Bap-Notch receptor (FIG. 13).

Collectively, these results support the claims that:
The method described here can regulate cellular gene expression programs based on artificial signaling platforms that are sensitive to Aβ inputs (oligomers, fibrils, and plaques).
Selective sensitivity/responsiveness to Aβ is programmable based on chosen recognition motifs.
   Recognition of vascular versus parenchymal amyloid is certainly conceptualized herein.
   Gant-Notch and Don-Notch do not potently respond to medium-supplemented Aβ42, whereas Bap-Notch does.
   Selectivity for Aβ42 versus Aβ40 is feasible.
Aβ-sensitive artificial receptors can be used to modulate gene expression in a variety of cell types, ranging from pluripotent stem cells, primary marrow-derived stem cells, astrocytes, and immortalized fibroblasts.
Diverse classes of transgenes can be regulated using this platform, for a similarly diverse suite of applications.
   Reporter genes—useful for tracking Aβ-interactive histories of cells.
   Putative neuroprotective or neurodegenerative gene variants—useful for mechanistic dissection of glia phenotypes in Aβ-associated niches.
   Potentially therapeutic factors—the work opens the possibility to program cells to localize therapeutic transgene expression, which can be deployed in the context of in situ gene therapy to native CNS cells or as a stem cell-based, engineered cell transplantation strategy.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims

### VIII. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,816,567
U.S. Pat. No. 4,867,973
U.S. Pat. No. 4,938,948
U.S. Pat. No. 5,021,236
U.S. Pat. No. 5,141,648
U.S. Pat. No. 5,196,066
U.S. Pat. No. 5,563,250
U.S. Pat. No. 5,565,332
U.S. Pat. No. 5,856,456
U.S. Pat. No. 5,880,270
U.S. Pat. No. 6,485,982
“Antibodies: A Laboratory Manual,” Cold Spring Harbor Press, Cold Spring Harbor, NY, 1988.
Abbondanzo et al.,Am. J. Pediatr. Hematol. Oncol. 12(4): 480-489, 1990.
Atagi et al., J. Biol. Chem. 290, 26043-26050 (2015).
Allred et al., Arch. Surg. 125(1), 107-113: 1990.
Atherton et al., Biol. of Reproduction 32: 155-171, 1985.
Bagheri-Mohammadi, Cell Tissue Bank. 22, 339-353 (2021).
Baik et al., Cell Metab. 30, 493-507.e6 (2019).
Bailey et al., J. Biol. Chem. 290, 26033-26042 (2015).
Barzon et al., Euro Surveill. 11:21(32), 2016.
Beltramello et al., Cell Host Microbe 8:271-283, 2010.
Blurton-Jones et al., Proc. Natl. Acad. Sci. U.S.A. 106, 13594-13599 (2009).
Brown et al., J. Immunol. Meth. 130(1): 111-121, 1990.
Butterfield & Halliwell, Nat. Rev. Neurosci. 20, 148-160 (2019).
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Capaldi et al., Biochem. Biophys. Res. Comm. 74(2):4 25-433, 1977.
Chen & Blurton-Jones, Stem Cells 30, 2612-2618 (2012).
De Jager et al., Semin. Nucl. Med. 23(2): 165-179, 1993.
Dholakia et al., J. Biol. Chem. 264:20638-20642, 1989.
Diamond et al., J. Virol. 77: 2578-2586, 2003.
Doolittle and Ben-Zeev, Methods Mol. Biol. 109:215-237, 1999.
Duffy et al., N. Engl. J. Med. 360: 2536-2543, 2009.
Elder et al. Infections, infertility and assisted reproduction. Part II: Infections in reproductive medicine & Part III: Infections and the assisted reproductive laboratory. Cambridge UK: Cambridge University Press; 2005.
Gefter et al., Somatic Cell Genet. 3:231-236, 1977.
Gornet et al., Semin Reprod Med. 34(5): 285-292, 2016.
Gratuze et al.,Mol. Neurodegener. 13, 66(2018 ).
Guerreiro et al., N. Engl. J. Med. 368, 117-127 (2013).
Gulbis and Galand, Hum. Pathol. 24(12): 1271-1285, 1993.
Guttenplan et al. Nature 599: 102-107 (2021).
Guttikonda et al. Nat. Neurosci. 24: 343-354 (2021).
Halfon et al., PLoS ONE 5(5): e10569, 2010
Hessell et al., Nature 449: 101-104, 2007.
Jay et al., J. Neurosci. 37, 637-647 (2017).
Jonsson et al., N. Engl. J. Med. 368, 107-116 (2013).
June & Sadelain, N. Engl. J. Med. 379, 64-73 (2018).
Khatoon et al., Ann. of Neurology 26: 210-219, 1989.
Kim et al., Nat. Comms 13(6581), (2022).
King et al., J. Biol. Chem. 269: 10210-10218, 1989.
Knopman et al., Nat. Rev. Dis. Prim. 7, 33(2021 ).
Kohler and Milstein, Eur. J. Immunol. 6: 511-519, 1976.
Kohler and Milstein, Nature 256: 495-497, 1975.
Kyte and Doolittle, J. Mol. Biol. 157(1): 105-132, 1982.
Leng et al., Nat. Neurosci. 25 : 1528-1542, 2022.
Liddelow et al., Nature 541 : 481-487 (2017).
Lim & June, Cell 168, 724-740 (2017).
Liu et al., World J. Stem Cells 12, 787-802 (2020).
Luca et al., Science (80-.). 355, 1320 LP- 1324(2017 ).
Mansuy et al., Lancet Infect Dis. 16(10): 1106-7, 2016.
Marsh et al., Stem cell reports 8, 235-248 (2017).
Morsut et al., Cell 164, 780-91 (2016).
Nagahara et al., Nat. Med. 15, 331-337 (2009).
Nagahara et al., J. Neurosci. 33, 15596-15602 (2013).
Nagahara et al., Gene Ther. 25, 104-114 (2018).
Nagahara & Tuszynski, Nat. Rev. Drug Discov. 10, 209-219 (2011).
Nakamura et al., In: Enzyme Immunoassays: Heterogeneous and Homogeneous Systems, Chapter 27, 1987.
O'Shannessy et al., J. Immun. Meth. 99: 153-161, 1987.
Persic et al., Gene 187: 1, 1997
Potter and Haley, Meth. Enzymol. 91: 613-633, 1983.
Purpura et al., Lancet Infect Dis. 16(10): 1107-8, 2016.
Remington's Pharmaceutical Sciences, 15th Ed., 3: 624-652, 1990.
Roybal et al., Cell 167, 419-432.e16 (2016a).
Roybal et al., Cell 164, 770-9 (2016b).
Salloway et al., JAMA Neurol. 79, 13-21 (2022).
Shen et al., J. Neurosci. Res. 95, 2420-2429 (2017).
Singh et al., J. Neuroinflammation 19(206) (2022).
Sperling et al., Lancet. Neurol. 11, 241-249 (2012).
Szymczak-Workman et al., bioRxiv; 0; doi: 10.1101/2021.10.20.465173v1 (2007).
Tan et al., Front. Biosci. (Schol. Ed). 4, 1402-1412 (2012).
Tang et al., J. Biol. Chem., 271: 28324-28330, 1996.
Tolar et al., Alzheimers. Res. Ther. 12, 95(2020 ).
Tönnies & Trushina, J. Alzheimers. Dis. 57, 1105-1121 (2017).
Ulrich et al., Neuron 94, 237-248 (2017).
Wang et al., Cell 160, 1061-1071 (2015).
Wawrzynczak & Thorpe, In: Immunoconjugates, Antibody Conuugates In Radioimaging And Therapy Of Cancer, Vogel (Ed.), NY, Oxford University Press, 28, 1987.
Xiong et al., Sci. Transl. Med. 13, (2021).
Yeh et al., Neuron 91, 328-340 (2016).
Yu et al., J Immunol Methods 336: 142-151, 2008.
Zhao et al., Neuron 97, 1023-1031.e7 (2018).
Zhu et al., Cell 185, 1431-1443 (2022).

1. An engineered cell expressing (a) a synthetic receptor expressed on the engineered cell's surface and comprising:
(1) an extracellular domain which serves as a recognition motif capable of engaging Aβ;
(2) a combination juxtamembrane/transmembrane domain which anchors the receptor in the cell membrane and contains protease cleavage sites that are exposed after receptor conformational changes take place in response to Aβ binding; and
(3) an intracellular domain that, upon Aβ binding and subsequent protease-based cleavage of the receptor, is untethered from the receptor, and
(b) a protein that is operably linked to a control sequence that is activated upon receptor binding to Aβ.

2. The engineered cell of claim 1, wherein the synthetic receptor comprises transmembrane domain, such as one selected from Notch4, SORT1, Notch1, PCDHGC3, APP, CLSTN1, NOTCH2, NOTCH3, CLSTN2, NECTIN1, and EPCAM, and a juxtamembrane domain, such as one selected from NRG1, CSF1R, NOTCH3, NOTCH1, KCNE3, AGER, NOTCH4, PTPRF, PTPRM, NOTCH2, NRG2, LRP1B, PTPRF, JAG2, KL, EPHA4, PTPRK, CDH5, NOTCH1 and DAG1.

3. The engineered cell of claim 1, wherein the sense and response platform is synNotch, such as where a Notch extracellular domain is replaced with a heterologous recognition motif and the Notch intracellular domain is replaced with a selected transcription factor, such as Ga14VP64, or the tetracycline transactivator or a mammalian-derived transcriptional domain, such as a zinc finger transcription factor.

4. The engineered cell of claim 1, wherein the sense and response platform is Synthetic Intramembrane Proteolysis Receptor (SNIPR).

5. The engineered cell of claim 1, wherein:
(i) the cell is a fibroblast, a mesenchymal stem cell, an astrocyte, a microglial cell, a neuron, a neural stem cell, a macrophage, a T cell or a microvascular endothelial cell;
(ii) the extracellular domain is an antigen variable domain, such as located in a nanobody or single chain Fv (scFv); and/or
(iii) the extracellular domain is an scFv is derived from the variable domain of an antibody selective for parenchymal versus vascular Aβ, an antibody selective for fibrillar Aβ or higher ordered aggregates, an antibody selective for Aβ plaques, or an antibody selective for fibrillar Aβ or higher ordered aggregates.

6. The engineered cell of claim 1, wherein the protein is a detectable marker protein, such as wherein the detectable marker protein is Green Fluorescent Protein, mCherry, Blue fluorescent protein, infrared fluorescent protein, mCardinal, firefly luciferase, nanoluciferase, a BRET reporter (e.g., lumifluor), a FRET reporter, secreted alkaline phosphatase, gaussian luciferase, or a MRI reporter (OATP1B3 transporter).

7. The engineered cell of claim 1, wherein the protein is a therapeutic protein.

8. The engineered cell of claim 7, wherein the therapeutic protein is brain-derived neurotrophic factor (BDNF), presenilin, anti-inflammatory proteins such as interleukin-10, transforming growth factor β1 (TGFβ1), fibroblast growth factor, native inhibitors of interleukin-1, (e.g., IL1Ra) interleukin-6, or tumor necrosis factor (e.g., sTNFR1) or biologic neutralizing factors (canakinumab, ziltivekimab, infliximab, adalimumab); inhibitors of complement or a cascade factor; an amyloid clearing factor (neprilysin, anti-Ab antibody); or an anti-tau factor (e.g., bepranemab).

9. The engineered cell of claim 1, wherein the protein is a protein that can promote an Alzheimer's Disease phenotype in said cell.

10. The engineered cell of claim 9, wherein the protein that can promote an Alzheimer's Disease phenotype in said cell is TREM2, CD33, APOE, SERPINA3, CLU, BIN1, SORL1, ABCA7, ADAM10, APP, PSEN1, or IGHG3.

11. A method of preventing the onset of, inhibiting the progression of or treating Alzheimer's Disease (AD) comprising administering to a subject in need thereof an engineered cell according to claim 1.

12. The method of claim 11, wherein the subject has been diagnosed with AD.

13. The method of claim 12, wherein the subject has been diagnosed by behavioral testing or by a brain scan.

14. The method of claim 11, wherein the subject has been determined to be at elevated risk of developing AD as compared to a populational average.

15. The method of claim 14, wherein the subject has been determined to be at elevated risk of developing AD as compared to a populational average by genetic testing or by familial history.

16. The method of claim 11, wherein the subject is over 40 years of age, over 50 years of age, over 60 years of age, over 70 years of age, or over 80 years of age.

17. The method of claim 11, further comprising administering said engineered cell to said subject more than once.

18. The method of claim 16, wherein the subject has been administered said engineered cell 2, 3, 4, 5, 6, 7, 8, 9, 10 15, or 20 times.

19. The method of claim 11, wherein the subject is administered said engineered cell every week, every two weeks, every three weeks, month, every other month, every three months, every four months, every six months or every year.

20. The method of claim 11, wherein the subject is treated with at least a second anti-AD therapy that reduces risk of developing AD and/or reduces symptoms of AD.

21. A method of assessing or monitoring amyloid accumulation in subject comprising administering to said subject an engineered cell according to claim 1.

22. The method of claim 21, wherein the subject has been diagnosed with AD.

23. The method of claim 22, wherein the subject has been diagnosed by behavioral testing or by a brain scan.

24. The method of claim 21, wherein the subject has been determined to be at elevated risk of developing AD as compared to a populational average.

25. The method of claim 24, wherein the subject has been determined to be at elevated risk of developing AD as compared to a populational average by genetic testing or by familial history.

26. The method of claim 21, wherein the subject is over 40 years of age, over 50 years of age, over 60 years of age, over 70 years of age, or over 80 years of age.

27. The method of claim 21, further comprising administering said engineered cell to said subject more than once.

28. The method of claim 26, wherein the subject is administered said engineered cell 2, 3, 4, 5, 6, 7, 8, 9, 10 15, or 20 times.

29. The method of claim 21, wherein the subject is administered said engineered cell every week, every two weeks, every three weeks, month, every other month, every three months, every four months, every six months or every year.

30. The method of claim 21, further comprising treating said subject with an anti-AD therapy that reduces risk of developing AD and/or reduces symptoms of AD.

31. One or more expression vectors expressing (a) a synthetic receptor expressed on the engineered cell's surface and comprising:
(1) an extracellular domain which serves as a recognition motif capable of engaging Aβ;
(2) a combination juxtamembrane/transmembrane domain which anchors the receptor in the cell membrane and contains protease cleavage sites that are exposed after receptor conformational changes take place in response to Aβ binding; and
(3) an intracellular domain that, upon Aβ binding and subsequent protease-based cleavage of the receptor, is untethered from the receptor, and
(b) a protein that is operably linked to a control sequence that is activated upon receptor binding to Aβ.

32. The expression vector(s) of claim 31, wherein the synthetic receptor comprises transmembrane domain, such as one selected from Notch4, SORT1, Notch1, PCDHGC3, APP, CLSTN1, NOTCH2, NOTCH3, CLSTN2, NECTIN1, and EPCAM, and a juxtamembrane domain, such as one selected from NRG1, CSF1R, NOTCH3, NOTCH1, KCNE3, AGER, NOTCH4, PTPRF, PTPRM, NOTCH2, NRG2, LRP1B, PTPRF, JAG2, KL, EPHA4, PTPRK, CDH5, NOTCH1 and DAG1.

33. The expression vector(s) of claim 31, wherein the sense and response platform is synNotch, such as where a Notch extracellular domain is replaced with a heterologous recognition motif and the Notch intracellular domain is replaced with a selected transcription factor, such as wherein the selected transcription factor is Ga14VP64, the tetracycline transactivator, or a mammalian-derived transcriptional domain, such as a zinc finger transcription factor.

34. The expression vector(s) of claim 31, wherein the sense and response platform is Synthetic Intramembrane Proteolysis Receptor (SNIPR).

35. The expression vector(s) of claim 31, wherein the extracellular domain is:
(i) an antigen variable domain is located in a nanobody or single chain Fv (scFv); and/or
(ii) an scFv is derived from the variable domain of an antibody selective for parenchymal versus vascular Aβ, an antibody selective for fibrillar Aβ or higher ordered aggregates, an antibody selective for Aβ plaques, or an antibody selective for fibrillar Aβ or higher ordered aggregates.

36. The expression vector(s) of claim 31, wherein the protein is a detectable marker protein, such as wherein the detectable marker protein is Green Fluorescent Protein, mCherry, Blue fluorescent protein, infrared fluorescent protein, mCardinal, firefly luciferase, nanoluciferase, a BRET reporter (e.g., lumifluor), a FRET reporter, secreted alkaline phosphatase, gaussian luciferase, or a MRI reporter (OATP1B3 transporter).

37. The expression vector(s) of claim 31, wherein the protein is a therapeutic protein.

38. The expression vector of claim 37, wherein the therapeutic protein is brain-derived neurotrophic factor (BDNF), presenilin, anti-inflammatory proteins such as interleukin-10, transforming growth factor β1 (TGFβ1), fibroblast growth factor, native inhibitors of interleukin-1, (e.g., IL1Ra) interleukin-6, or tumor necrosis factor (e.g., sTNFR1) or biologic neutralizing factors (canakinumab, ziltivekimab, infliximab, adalimumab); inhibitors of complement or a cascade factor; an amyloid clearing factor (neprilysin, anti-Aβ antibody); or an anti-tau factor (e.g., bepranemab).

39. The expression vector(s) of claim 31, wherein the protein is a protein that can promote an Alzheimer's Disease phenotype in said cell.

40. The expression vector(s) of claim 39, wherein the protein that can promote an Alzheimer's Disease phenotype in said cell is TREM2, CD33, APOE, SERPINA3, CLU, BIN1, SORL1, ABCA7, ADAM10, APP, PSEN1, or IGHG3.

41. A method of preventing the onset of, inhibiting the progression of or treating Alzheimer's Disease (AD) comprising administering to a subject in need thereof a vector or vectors according to claim 31.

42. The method of claim 41, wherein the subject has been diagnosed with AD.

43. The method of claim 42, wherein the subject has been diagnosed by behavioral testing or by a brain scan.

44. The method of claim 41, wherein the subject has been determined to be at elevated risk of developing AD as compared to a populational average.

45. The method of claim 44, wherein the subject has been determined to be at elevated risk of developing AD as compared to a populational average by genetic testing or by familial history.

46. The method of claim 41, wherein the subject is over 40 years of age, over 50 years of age, over 60 years of age, over 70 years of age, or over 80 years of age.

47. The method of claim 41, further comprising administering said expression vector(s) to said subject more than once, such as 2, 3, 4, 5, 6, 7, 8, 9, 10 15, or 20 times, and/or wherein the subject is administered said expression vector(s) every week, every two weeks, every three weeks, month, every other month, every three months, every four months, every six months or every year.

48. The method of claim 41, wherein the subject is treated with at least a second anti-AD therapy that reduces risk of developing AD and/or reduces symptoms of AD.

49. The method of claim 41, wherein the expression vector(s) is/are viral vectors, such as an AAV vector, a lentiviral vector and an HSV vector.

50. The method of claim 41, wherein the expression vector(s) is/are non-viral vectors, such a non-viral vector delivered in or with a lipid nanoparticle, porous silicon nanoparticle, polymer nanoparticles, or gold nanoparticles, dendrimers, carbon nanotubes.
